(19) United States
(12) Patent Application Publication
(10) Pub. No.: US 2015/0376664 A1
(43) Pub. Date: Dec. 31, 2015

(54) METHOD AND SYSTEM FOR SYNTHESIZING TAXOL FROM CORYLUS AVELLANA TISSUE CULTURE

(71) Applicants: Faezeh Ghanati, Tehran (IR); Masoumeh Safari, Tehran (IR); Abazar Hajnorouzi, Tehran (IR)

(72) Inventors: Faezeh Ghanati, Tehran (IR); Masoumeh Safari, Tehran (IR); Abazar Hajnorouzi, Tehran (IR)

(21) Appl. No.: 14/848,913

(22) Filed: Sep. 9, 2015

(51) Int. Cl. C12P 17/02; C12M 1/24; C12M 1/42; G01N 33/50; C12M 1/34

(52) U.S. Cl. CPC C12P 17/02; G01N 33/5097; C12M 41/00; C12M 35/02; C12M 35/04; C12M 23/08

(57) ABSTRACT

A spherical bioreactor system and a method of synthesizing taxol from hazel (Corylus avellana) cell culture are disclosed. The bioreactor has a round bottomed flask fitted with two piezoelectric transducers connected to an audio amplifier, a voltage controlled oscillator, an integrator and a lock in amplifier. For synthesizing taxol, A rapid growing cell line is established from the hazel seeds collected from a cell line culture in a modified MS medium. A suspension culture is established from the rapid growing cell line. The preliminary studies are conducted for estimating an ultrasound dosage and exposure time for treating the suspension culture. The cells are harvested, washed and transferred to a fresh culture media for 1 week. The cells and the culture media are analyzed for molecular and biochemical parameters. The cells are dried for extracting intracellular and extracellular taxols.

### SPONSORSHIP STATEMENT

This application is financially sponsored for international filing by the IRANIAN NATIONAL SCIENCE FOUNDATION (INSF).

### BACKGROUND

1. Technical Field

The embodiments herein generally relates to tissue culture and tissue engineering. The embodiments herein particularly relate to an ultrasound bioreactor. The embodiments herein also relates to a method and ultrasound bioreactor for producing or synthesizing phytochemical using ultrasound.

2. Description of the Related Art

Phytochemicals are chemical compounds that occur naturally in plants. The phytochemicals are responsible for color and other organoleptic properties such as the deep purple color of blueberries and smell of garlic. The phytochemicals have biological significance for example carotenoids or flavonoids.

Phytochemicals have been considered possible drugs for millennium. For example willow tree leaves are prescribed to abate fever. Salicin has anti-inflammatory and pain relieving properties. Salicin is extracted from the bark of white willow tree and later synthetically produced to become the staple, over the counter drug aspirin. Some phytochemicals with physiological properties may be elements rather than complex organic molecules. For example selenium which is abundant in many fruits and vegetables is dietary mineral involved in major metabolic pathways including thyroid hormone metabolism and immune function. Particularly it is an essential nutrient and cofactor for the enzymatic synthesis of glutathione, an endogenous antioxidant.

Taxol (generic name, Paclitaxel) is a diterpen alkaloid which was first isolated from the bark of the Pacific Yew tree (Taxus brevifolia) in 1971. It is an anti-cancer drug which has been approved by FDA for the treatment of breast cancer, lung cancer and ovarian cancer as well as AIDS related Kaposi's sarcoma. The principal targets of taxol are microtubules of essential components of the cytoskeleton. The mechanism by which taxol or paclitaxel acts against rapidly proliferating cancer cell populations includes paclitaxel stabilizing microtubules, interrupting dynamic instability of microtubules and thereby promoting cellular arrest during mitosis (G2/M phase). The other taxanes (baccatin III, cephalomannine and 10-deacetyl baccatin) have been derived from Taxus species and fungal endophyte of yew tree.

Despite the use of taxol or paclitaxel as a cure for cancer the isolation of taxol from the bark of Pacific yew trees is time consuming, expensive and ineffective. The tree is slow growing and two to four of it is required to obtain enough taxol to treat one patient. Hence the natural resource for extracting taxol is being threatened day by day to the destructive collection of Taxus bark for taxol.

The taxol is well known anti-cancer drug. The taxol is isolated from the bark of the yew tree (Taxus sps). Despite being cure for cancer, the isolation of taxol from the bark of Pacific yew trees is time consuming, expensive and indefinitive. The tree is slow growing and two to four of it is required to obtain enough taxol to treat one patient. Hazel (Corylus avellana) is widely available and hazel cells grow faster in vivo and are easier to cultivate than Pacific yew trees.

Recently the presence of taxanes including palitaxel, 10-deacetylbaccatin III, baccatin III, paclitaxel C and 7-epipaclitaxel is confirmed in the shells and leaves of hazel plants. When compared to Yew trees, the hazel (Corylus avellana) is widely available and hazel cells grow faster in vivo and are easier cultivated in vitro than yew. Iranian provinces such as Urmia, Gorgan, Gillan and Mazandarn are the important regions for growth of the hazel tree. Iran with an annual production of 14,299 tons is the seventh hazelnut producer in the world. However similar to Yew tree, the hazel trees should be protected against taxol extraction. Plant cell and tissue cultures hold great promise for controlled production of myriad of useful secondary metabolites on demand. Plant cell cultures can serve as a renewable source of valuable secondary metabolites.

A bioreactor is referred to a device or system meant for growing cell or tissues in the context of cell culture. These devices are being developed for use in tissue engineering or bio-chemical engineering. The bioreactor is also referred to any manufacturing or engineering device or system that supports a biologically active environment. A bioreactor is a vessel in which a chemical process is carried out which involves organisms or biologically active substances derived from such organisms. The process can be aerobic or anaerobic. The bioreactors are commonly cylindrical, ranging in size from liters to cubic meters. The bioreactors are often made of stainless steel or sometimes glass.

Bioreactors offer several advantages for culturing cells and tissues compared with simple tissue-flask and Petri-dish culture systems, notably, the ability to provide mechanical forces influencing tissue development and to achieve better control over culture conditions. Designs of bioreactors that are currently available for cultivating tissue-engineered constructs are based primarily on hydrostatic pressure (e.g., dynamic compression), hydrodynamic stress at low shear rates (e.g., perfusion systems), rotating bioreactors, wavy-wall bioreactors, and conventional spinning flasks. Central to a successful tissue engineering strategy to grow functional tissue equivalents is the establishment of a bioreactor or a bio-processing unit that maintains cells seeded on biodegradable scaffolds and provides essential gas and nutrient transport between the cells and the culture media, as well as the mechanical stimuli necessary to promote extracellular matrix synthesis.

A variety of vessels and methods have been developed over the years to carry out chemical, biochemical and/or biological processing. Stainless steel fermentation vessels of several hundreds of thousands liters are not uncommon for the growth of microorganisms that produce enzymes or secondary metabolites. The methods include batch, fed-batch, continuous or semi-continuous perfusion. Gradually, more challenging cultures such as mammalian, insect or plant cells have been adapted for growth in fermentation vessels using highly specialized media. Although the design of these vessels differs, but they have several common features. The cells are kept in suspension by rotating stirring blades placed vertical in the vessel, and gas exchange is facilitated by injection of air, oxygen or carbon dioxide.

There are several drawbacks in the design of the conventionally used bioreactors. The shearing forces that are introduced through the stirring blades and the cavitations of miniscule air bubbles are detrimental to more sensitive cell types or organisms. Also, these vessels have to be rigorously cleaned between production runs to prevent cross-contamination, which is time consuming and needs to be validated for individual cultures. Furthermore, the use of stirred fermenters requires highly trained operators. The cost price for stirred fermenters is high across the whole size range and therefore they are used repeatedly over long periods of time, thus increasing infection risks as a result of mechanical failures. Perhaps most significantly, the optimization of culture conditions for stirred fermenters in a small scale cannot be transferred in a linear way to commercial scale production. For example, the fluid dynamics, aeration, foaming and cell growth properties change with an order of magnitude when the scale increases. In addition, for more delicate cell types or organisms, a large scale stirred fermentation vessel is not a viable device, even when more subtle stirring techniques such as airlift fermenters are used.

Ultrasound is an oscillating sound pressure wave with a frequency greater than the upper limit of the human hearing range. Ultrasound is thus not separated from normal (audible) sound by differences in physical properties only by the fact that humans cannot hear it. Ultrasound is used in many different fields. Ultrasonic devices are used to detect objects and measure distances. Ultrasonic imaging (sonography) is used in both veterinary medicine and human medicine. In the non destructive testing of products and structures, ultrasound is used to detect invisible flaws. Ultrasound has been widely applied in medicine, biology, diagnosis and therapy. The biological effects of ultrasound have received considerable attentions. High intensity ultrasound is well known to be destructive to biological materials, disrupting the cell membranes and deactivating biological molecules such as enzymes and DNA. Low intensity ultrasound on the other hand has shown a range of sub-lethal biological effects that are of potential significance in biotechnology.

Bioreactors are generally used to grow cells that mach certain specifications. In standard bioreactors, tissue samples need to be removed from within the device periodically, sliced up and tested under the microscope that is time-consuming and labor intensive process. To overcome the aforesaid problems ultrasound is used for biokinetics and monitoring cell status by mathematical models. Such reactors usually applied for human tissue engineering purposes and have not been introduced for pharmaceutical research. The ultrasound stimulates cells for phytochemical production. As the ultrasound is not destructive to the cells and cells are sub-cultured. The produced phytochemical is harvested from culture medium continuously.

Hence there is a need for a method for production of phytochemical, specifically taxol under controlled conditions. Also there is a need for a ultrasound bioreactor for the production of taxol, such that the culture medium is free from microbes and insects. Further there is a need for a automated control of cell growth and rational regulation of metabolite processes which reduce labor costs and increase productivity.

The above mentioned shortcomings, disadvantages and problems are addressed herein and which will be understood by reading and studying the following specification.

### OBJECTIVES OF THE EMBODIMENTS

The primary objective of the embodiment herein is to synthesize taxol from plant tissue culture of Corylus avellana.

Another object of the embodiment herein is to provide ultrasound bioreactor for synthesis of taxol from plant tissue culture controlled condition.

Yet another object of the embodiment herein is to investigate the effects of ultrasounds on the medicinal plant Corylus avellana suspension culture and increase in the taxol production.

Yet another object of the embodiment herein is to synthesize taxol in ultrasound bioreactor free from microbial contamination and insects.

Yet another object of the embodiment herein is to provide an ultrasound bioreactor with automated control of cell growth and rational regulation of metabolite processes which reduces labor costs and increases yield of taxol.

Yet another object of the embodiment herein is to provide an ultrasound bioreactor which has no adverse on cell viability, growth and membrane integrity.

Yet another objective of the embodiment herein is to provide an ultrasound bioreactor which has positive effect on biomass yield and increase taxol biosynthesis.

Yet another objective of the embodiment of the embodiment herein is to establish fast growing cell line of hazel Corylus avellana which serves as renewable source of taxol.

Yet another objective of the embodiment of the embodiment herein is to produce low intensity ultrasound of definite frequency by two piezoelectric transducers.

Yet another objective of the embodiment of the embodiment herein is to induce production of reactive oxygen species (ROS) which are signaling molecule stimulating the gene expression and promote taxol production molecular pathway.

Yet another object of the embodiment herein is to provide an ultrasound bioreactor which is not destructive to the cells and the cells are sub-cultured for producing drugs to be harvested from culture medium continuously.

These objects and the other advantages of the embodiments herein will become readily apparent from the following detailed description taken in conjunction with the accompanying drawings.

### SUMMARY

The various embodiments herein provide a piezoelectric, low intensity ultrasound bioreactor to investigate the effects of ultrasound on medicinal plant (Corylus avellana) tissue suspension culture for taxol production. The plant tissue culture for the production of taxol has many advantages. The useful phytochemical (taxol) is produced under controlled conditions independent of the external factors in which the plant/tree grows. The cultured cells and the medium is free from microbes and insects. The cells of any plants, tropical or alpine are easily multiplied to yield specific metabolites. The automated control of cell growth and regulation of metabolite processes reduces labor costs and increases productivity.

According to one embodiment herein, a spherical bioreactor system for synthesizing taxol from hazel (Corylus avellana) cell culture is provided. The system comprises a round bottomed flask, a plurality of piezoelectric transducers, an audio amplifier, a voltage controlled oscillator, integrator and a lock in amplifier. The piezoelectric transducers comprises a series of four pairs of piezoelectric crystals fixed together. The piezoelectric crystals are configured to induce a resonance in a circuit of system. The audio amplifier is configured to supply electrical power to the piezoelectric transducers. The audio amplifier is configured to amplify low power audio signals to a suitable level. The voltage controlled oscillator is configured to supply electrical power and generate ultra sound signals in the spherical bioreactor system. The voltage controlled oscillator transfers or supplies power to the audio amplifier. The integrator is configured to cumulate the input signal into an output signal. The integrator output signal is a time integral of the output signal. The lock in amplifier is configured to extract signals with a known carrier wave from a noisy environment. The oscillation frequency of the voltage controlled oscillator is controlled by an input voltage input. The input voltage estimates an instantaneous oscillation frequency. The voltage controlled oscillator modulates signals applied to control the input thereby causing frequency modulation or phase modulation. The system produces continuous ultrasound waves at preset frequency with different power output levels.

According to one embodiment herein, the round bottomed flask has a volume of 100 ml and the round bottomed flask is preferably made of glass.

According to one embodiment herein, the pluralities of piezoelectric transducers are two. The piezoelectric transducers have a length of 12.1 mm and the piezoelectric transducers have a diameter of 20 mm. The two piezoelectric transducers are mounted 180° degrees apart near a central portion of the round bottomed flask. The piezoelectric transducers are mounted on the round bottomed flask using a dry epoxy material.

According to one embodiment herein, the system produces ultrasound waves of 29 kHz with different levels of power output. The spatial peak intensity (Isp) of the system is in a range of 4-455 mW/cm³.

According to one embodiment herein, the method of synthesizing taxol in a spherical bioreactor from hazel (Corylusavellana) cell culture, comprises the following steps. The first step is obtaining the hazel (Corylusavellana) seeds tissue for cell line culture. A rapid growing cell line culture or callus culture is established from hazel seed tissue in a modified Murashige and Skoog medium (MS medium). A suspension culture is established from the cell line culture. The suspension culture is established from hazel seeds after growing the cell line culture or callus culture in modified the Murashige and Skoog medium (MS medium). The cells in a logarithmic growth phase is transferred along with culture media to a spherical ultrasonic bioreactor. The preliminary studies are conducted for estimating an ultrasound dosage and time for treating suspension culture with ultrasound signals. The cell line culture in the spherical ultrasonic bioreactor is treated or irradiated with the ultrasound of preset power levels for preset time periods. The cells are harvested after the ultrasound exposure. The harvested cells are washed and transferred to a fresh culture media for 1 week. The cells and culture media are analyzed or molecular and biochemical parameters. The cell suspension culture of the hazel (Corylus avellana) is analyzed for the molecular and biochemical parameters. The molecular and biochemical parameters are taxane production analysis. The cells are dried for extracting taxol. The intracellular and extracellular taxanes are extracted from the taxols.

According to one embodiment herein, a modified Murashige and Skoog (MS) medium comprises ammonium nitrate (NH₄NO₃) at a quantity of 1650 mgL⁻¹, monopotassium phosphate (KH₂PO₄) at a quantity of 170 mg L⁻¹, calcium chloride (CaCl₂) at a quantity of 332.02 mg L⁻¹, magnesium sulfate (MgSO₄) at a quantity of 180.54 mgL⁻¹, ferric-ethylenediamine-tetra-acetic acid (Fe-EDTA) at a quantity of 36.70 mgL⁻¹, boric acid (H₃BO₃) at a quantity of 6.20 mgL, cupric sulphate pentahydrate (CuSO₄5H₂O) at a quantity of 0.025 mgL⁻¹, manganese(II) sulfate monohydrate (MnSO₄H₂O) at a quantity of 16.90 mg L⁻¹, sodium molybdate dehydrate (Na₂MoO₄ 2H₂O) at a quantity of 0.25 mg L⁻¹, zinc sulphate heptahydrate (ZnSO₄7H₂O) at a quantity of 8.60 mg L⁻¹, potassium iodide (KI) at a quantity of 0.83, Cobalt(II) chloride hexahydrate (CoCl₂6H₂O) at a quantity of 0.025 mg L⁻¹, and supplemented with 3% sucrose, and 1 mg L⁻¹ 4-dichlorophenoxyacetic acid and 0.5 mg L⁻¹ of benzyladenine. The pH of the modified Murashige and Skoog medium (MS medium) is 5.5.

According to one embodiment herein, the modified Murashige and Skoog medium (MS medium) for suspension culture of hazel seeds further comprises a naphthalene acetic acid (NAA) at a quantity of 3 mg L⁻¹ and a indole-3-acetic acid (IAA) at a quantity of 3 mg L⁻¹.

According to one embodiment herein, 50 subculture of the cell line a suspension culture is established, and the cells in suspension culture are incubated at 25° C. in dark with a shaking or agitation at 110 rpm on shaker incubator. The cells are sub-cultured every 7 days.

According to one embodiment herein, the cells are exposed to ultrasound energy of preset intensities for preset time periods. The preset intensity level of ultrasound energy for irradiating cell suspension culture is within a range of 4 to 455 mW/cm³. The preset time duration of ultrasound radiation for cell suspension culture is within a range of 2 to 60 min.

According to one embodiment herein, the cells are harvested, cultured and transferred to a fresh culture media after exposure to ultrasound energy. The fresh culture media is a modified Murashige and Skoog medium (MS medium) for cell growth and taxol production. The cells are cultured for 1 week in the modified Murashige and Skoog medium (MS medium).

According to one embodiment herein, the taxane production is analyzed by high performance liquid chromatography-mass spectrometry (HPLC-MS).

According to one embodiment herein, the step of analysing the cells and culture media for molecular and biochemical parameters comprises hydrogen peroxide (H₂O₂) production analysis, enzyme and enzyme activity analysis, an agarose gel electrophoresis analysis, a taxane production analysis of cell suspension culture with and without exposure to ultrasound energy, a viability and growth analysis of the hazel (Corylus avellana) cells after subjecting the cells to ultrasound irradiation, analysis of the relation between duration of ultrasound sonication and production of iodine and analysis of the relation between the spherical Bessel function and luminescence.

According to one embodiment herein, the enzymes analysed are 1-deoxy-D-xylulose-5-phosphate reducto-isomerase (DXR) and phenylalanine ammonia lyase (PAL).

According to one embodiment herein, the hydrogen peroxide (H₂O₂) is produced by the cell line of hazel under preset control conditions in the spherical bioreactor. The hydrogen peroxide (H₂O₂) is a reactive oxygen species. The hydrogen peroxide (H₂O₂) promotes a production of a taxol or taxanes. The hydrogen peroxide (H₂O₂) is a signaling molecule for stimulating a gene expression and promotes taxol production molecular pathway.

According to one embodiment herein, the hazel cells are exposed to ultrasound irradiation for a time period of 20 minutes to achieve a highest 1-deoxy-D-xylulose-5-phosphate reductoisomerase (DXR) activity in a harvesting time of 6 hours. The hazel cells are exposed to ultrasound irradiation for a time period of 20 minutes to achieve a highest phenylalanine ammonia lyase (PAL) activity in a harvesting time of 24 hours.

According to one embodiment herein, the highest taxol content is produced after exposing the hazel cell culture to ultrasound irradiation for 20 minute. A dry weight of the taxol content produced is 6 mg/Kg, when the hazel cell culture is exposed to ultrasound irradiation for 20 minute.

According to one embodiment herein, the exposure of hazel cells to ultrasound in the ultrasound bioreactor has no adverse effects on cell viability, cell growth and cellular membrane integrity.

According to one embodiment herein, the exposure of hazel cells to ultrasound has positive effects on biomass yields and increased taxanes biosynthesis.

According to one embodiment herein, the intracellular and extracellular taxanes are taxol, 10-deacetyl baccatin III and baccatin III.

According to one embodiment herein, the extraction of cell-associated intracellular and extracellular taxol comprises the following steps. The cells are collected from the culture medium. The collected cells are washed. The cells are dried for a predetermined period at room temperature. The predetermined drying time is 5 minutes. The dried cells are powdered at room temperature. The powdered cells are dissolved in 10 mL methanol to obtain a solution. The solution of powdered cell in methanol is ultrasonicated or irradiated with ultrasounds for 40 min to obtain a homogenate. The homogenate is filtered. The filtrate is air dried and re-dissolved in a mixture of methylene chloride and water. The methylene chloride and water are mixed in a ratio of 1:1. The dissolved filtrate is centrifuged at 5000 rpm to obtain a supernatant and a pellet. The supernatant is eluted to obtain a methylene chloride phase. The methylene chloride phase is collected. The methylene chloride phase is air-dried and re-dissolved in a 250 mL methanol. The dissolved methylene chloride phase in methanol is filtered with a 0.45-mm syringe filter. The filtrate is subjected to a high-performance liquid chromatography (HPLC) analysis. The HPLC system is equipped with a C-18 column. The extracellular taxol (in the medium) with methylene chloride (1:1) is extracted in a separating funnel. The taxol is eluted at a flow rate of 1 mL min-1 methanol and water (45:55, v/v). The taxol is detected at 227 nm using an ultraviolet detector. The quantity of taxol produced is estimated by comparison of a retention time and peak area with that of a genuine standard.

According to one embodiment herein, taxol (generic name, paclitaxel) is a well known anticancer drug. Taxol is a diterpene alkaloid which is usually isolated from the bark of the yew tree (Taxus species). Despite the use of taxolas a cure for cancer, the isolation of taxol from the bark of the Pacific yew trees is time consuming, expensive and ineffective. The tree is slow growing and two to four of it is required to obtain enough taxol to treat one patient. Another tree hazel (Corylus avellana) is widely available and hazel cells grow faster in vivo and are easily cultivated in vitro than yew.

According to one embodiment herein, ultrasound has been widely applied in medicine and biology for diagnosis and therapy during many decades. The biological effects of ultrasound have received considerable attentions. High intensity ultrasound is well known to be destructive to biological materials, disrupting the cell membranes and deactivating biological molecules such as enzymes and DNA. Low-intensity ultrasound on the other hand has shown a range of sub-lethal biological effects that are of potential significance in biotechnology. Series of experiments are conducted using ultrasonic bath with different power densities, different periods and repetitions of exposure to ultrasound on hazel cells.

Low-intensity ultrasound is known as a physical elicitor. It has a various effects on biological pathways in the living cells including the increase of reactive oxygen species (ROS) production. The mechanical stress of ultrasound on cells suspended in liquid medium arises from two hydrodynamic events. The hydrodynamic events are acoustic cavitation and cavitation induced micro-streaming. These hydrodynamic events results in shear stress, subsequently producing ROS. The ROS affects the plant cells, such as causing damage to cell membranes, essential macromolecules such as proteins, DNA, and lipids. The ROS also function as signal molecule to trigger a series of cellular responses from expression of genes to produce taxanes. In the spherical ultrasound bioreactor, due to specificity of growth phase, exposure duration, and ultrasound dosage a definite amount of ROS are produced. The level of ROS produced in ultrasound bioreactor does not destruct the plant cells but is efficient enough to promote taxane production pathway.

According to one embodiment herein, a piezoelectric type, low intensity ultrasound generating apparatus (a spherical ultrasonic bioreactor) is designed in order to investigating the effects of ultrasounds on tissue culture of medicinal plant. Particularly the effect of ultrasound on suspension culture of hazel cells and the analysis is done for possibility of increase in taxol production.

According to one embodiment herein, the spherical ultrasound bioreactor is composed of two piezoelectric transducers (12.1 and 20 mm) in diameter and length respectively). The transducers are mounted 180° apart as near to the equator of the bottom of 100 ml flask. The transducers are mounted using a quick dry epoxy attached to both sides of bioreactor. Each transducer is composed of a series of 4 two piezo-ceramic crystals fixed together so that the circuit resonates and maximum power is delivered to flask. Power is supplied through a voltage controlled oscillator (Juya Electronic Co. Iran). The resonance frequency of the flask is determined by searching for peaks in the amplitude of the sound field in the reactor by tracking the phase difference between voltage and current of the transducer. The device produces continuous ultrasound waves at 29 kHz with different levels of power output. Acoustic calibration for the power and intensity of the device is carried out in degassed water in the tank, using a radiation force balance (Shrewbury Medical Co. UK) and the hydrophone method in the cubic chamber (PA124, Precision Acoustics Ltd Dorchester Dorset UK). The calibration range is 10 kHz-20 MHz, with a 25 mm sensor diameter. The measurements are carried out when the distance between head of hydrophone and the device is 1 cm. The real frequency and actual spatial peak intensities (Isp) of this ultrasonic unit are 29 KHz and 4-455 mW/cm³ respectively, fundamental frequency based on calibration certification test results. To find the appropriate exposure dosage a set of preliminary studies are conducted using different durations of 2 to 60 minutes and power intensities of 4-455 mW/cm³.

According to one embodiment herein, a fast growing cell line for hazel Corylus avellana is established. The cell line and cell culture serve as renewable source of taxanes. In the bioreactor the controlled production of taxol and other anti-cancer drugs is possible. The growth of the suspension culture of hazel cells is achieved under controlled conditions independent of climatic changes or soil changes, free from microbial infection and insects. The cells are multiplied to yield specific metabolites to gather with reduced labor costs and improved productivity. The taxanes are extracted from cultured plant tissue culture.

According to one embodiment herein, hazel cells are grown in suspension cultures in shake-flasks with the specific culture conditions and specific culture medium formulation. The growth of the cells is plotted against time and the appropriate time for application of ultrasound is decided. The cells are exposed to definite power density of ultrasound just for a short period (found by other sets of experiments). The cells are then transferred back to their batches in order to continue to growth and Taxol production. The ultrasound in bioreactor is applied in order to stimulate the cells for Taxol production and not for monitoring the cell status. This ultrasound bioreactor system is not destructive to the cells and they can be sub-cultured and their produced drugs be harvested from medium continuously.

According to one embodiment herein, a rapidly growing cell line is induced from the seeds of Iranian cultivar of Corylus avellana, which is cultivated in northern Iran. The callus is established in a modified MS medium without glycine and supplemented with 3 mg NAA/L, 3 mg ISS/L and 1 mg kinetin/L. Where 1-naphthalene acetic acid is NAA and indole-3-acetic acid is IAA respectively. The suspension culture is established by transferring 1 gram callus in 30 ml culture medium. The callus in culture medium is incubated at 25° C. in a dark environment on a rotary shaker with 110-120 rpm. The growth curve of the cells is plotted in order to find appropriate times for manipulation of the cells. The suspension is renewed every seven days. Further various strategies are employed to increase the production of taxol in hazel cell cultures, i.e. addition of different precursor, elicitation with different chemicals and physical elicitors (including magnetic fields and different types of ultrasounds) or combination of these strategies.

According to one embodiment herein, ultrasound is widely applied in medicine and biology for diagnosis and therapy during many decades. The results illustrates that short exposure of hazel cells to ultrasound enhances both extracellular and cell-associated taxol contents. The extracellular yield of taxol increases. This increase is attributed to ultrasound-induced temporary increase of membrane permeability that facilitated the release of paclitaxel into the medium. Further results illustrate that exposure of hazel cells to ultrasound in this bioreactor has no adverse effects on cell viability, cell growth and membrane integrity but also excreted beneficial effects on biomass yields and increase in taxol yield. The system has the potential to produce appropriate amounts of signaling molecules thereby regulate activities of stress related enzymes and expression of their genes example: catalase. The ultrasound bioreactor system also enhances the expression of phenylalanine ammonia lyase (PAL), a key enzyme involved in signaling transduction pathways resulted in paclitaxel production and 1-deoxy-D-xylulose-5-phosphatereductoisomerase (DXR), a key enzyme of paclitaxel production pathway.

According to one embodiment herein, a piezoelectric type, low intensity ultrasound generating apparatus (spherical ultrasonic bioreactor) is designed in order to investigate the effects of ultrasounds on cell suspension-culture of hazel cells and the possibility of increase of taxol production by them.

The spherical ultrasonic bioreactor is composed of two piezoelectric transducers (12.1 and 20 mm in diameter and length, respectively). The piezoelectric transducers are mounted 180 degrees apart as near to the equator of a round bottom 100 mL flask. The piezoelectric transducers are mounted on both sides of bioreactor using a quick dry epoxy. Each transducer is composed of a series of 4 two piezo-ceramic crystals fixed together so that resonate the circuit, thereby delivering maximum power to the flask. The electric power is supplied through a voltage-controlled oscillator. The resonant frequency of the flask is determined by analyzing the peaks in the amplitude of the sound field of the reactor by tracking the phase difference between voltage and current of the transducer. The spherical ultrasonic bioreactor produces continuous ultrasound waves at 29 kHz with different levels of power output. Acoustic calibration for the power and intensity of the device is carried out in degassed water in a tank, using a radiation force balance (Shrewsbury Medical Co, UK) and by the hydrophone method in the cubic chamber (PA124, Precision Acoustics Ltd, Dorchester, Dorset, UK, calibration range: 10 kHz-20 MHz, with a 25 mm sensor diameter). The power and intensity measurements are carried out when the distance between head of hydrophone and the device is 1 cm. The real frequency and actual spatial peak intensities (Isp) of this ultrasonic unit are, 29 kHz and 4-455 mW/cm³ respectively, based on fundamental frequency calibration certification test results. To find the appropriate exposure dosage, a set of preliminary studies are conducted using different durations ranging 2 to 60 min) and power intensities ranging 4-455 mW/cm3).

According to one embodiment herein, a rapid growing cell line is established from the seeds of Iranian cultivar of hazel (Corylus avellana), in a modified Murashige and Skoog (MS) medium comprising (mg L−1): ammonium nitrate (NH₄NO₃) 1650, monopotassium phosphate (KH₂PO₄) 170, calcium chloride (CaCl₂) 332.02, magnesium sulfate (MgSO₄) 180.54, ferric-ethylenediamine-tetra-acetic acid (Fe-EDTA) 36.70, boric acid (H₃BO₃) 6.20, cupric sulfate pentahydrate (CuSO₄ 5H₂O) 0.025, manganese(II) sulfate monohydrate (MnSO₄H₂O) 16.90, Sodium molybdate dehydrate (Na₂MoO₄2H₂O) 0.25, zinc sulfate heptahydrate (ZnSO₄ 7H₂O) 8.60, potassium iodide (KI) 0.83, Cobalt(II) chloride hexahydrate (CoCl₂6H₂O) 0.025, and supplemented with 3% sucrose, and 1 mg L−1 2,4-dichlorophenoxyacetic acid and 0.5 mg L−1benzyladenine at pH 5.5.

After 50 subcultures of the cell line, suspension cultures are established in a modified medium basically similar to the aforesaid but supplemented with 3 mg L−1-naphthalene acetic acid (NAA) and 3 mg L-lindole-3-acetic acid (IAA). The cells are incubated at 25° C. in dark with shaking at 110 rpm. The cells are sub-cultured every 7 days. The cells (in their logarithmic growth phase) together with their media are transferred in a spherical ultrasonic bioreactor and are exposed to ultrasound of definite power densities and periods. After ultrasound exposure, the cells are cultured, harvested, thoroughly washed, and transferred back to fresh media in order to allow cells to grow and complete taxol production for further 1 week. The extracted cells are also thoroughly washed and dried for the taxol extraction.

For extraction of cell-associated (intracellular) Taxol, the dried cells are powdered for 5 min at room temperature. The powder of cells is dissolved in 10 mL methanol and ultrasonicated for 40 min to obtain a homogenate. The homogenate is filtered. The filtrate is air dried and re-dissolved in a mixture of methylene chloride and water (1:1). The dissolved filterate is centrifuged at 5000 rpm. After centrifugation the methylenechloride phase is collected, air-dried and re-dissolved in 250 mL methanol and filtered with a 0.45-mm syringe filter before high-performance liquid chromatography (HPLC) analysis. Furthermore, extracellular Taxol (in the medium) is extracted with methylene chloride (1:1) in a separating funnel. The methylene chloride phase is collected, air dried, re-dissolved in 250 mL methanol and filtered with a 0.45-mm syringe filter for high-performance liquid chromatography (HPLC) analysis. The HPLC system (Knauer, Berlin, Germany) is equipped with a C-18 column (Perfectsil Target ODS3, 5 mm, 250×4.6 mm, MZ-Analysentechnik, Mainz, Germany). The taxol is eluted at a flow rate of 1 mL min⁻¹ methanol and water (45:55, v/v) and is detected at 227 nm using an ultraviolet detector (PDA, Berlin, Germany). The quantification of taxol is accomplished by comparison of retention time and peak area with genuine standard (Sigma, St. Louis, Mo., USA).

According to one embodiment herein, the cell suspension culture of the hazel (Corylus avellana) are analyzed for the following: (a) analysis of taxane production by high performance liquid chromatography-mass spectrometry (HPLC-MS), (b) hydrogen peroxide (H₂O₂) production analysis, (c) Enzyme and enzyme activity analysis (1-deoxy-D-xylulose-5-phosphate reductoisomerase (DXR) and phenylalanine ammonia lyase (PAL) and agarose gel electrophoresis analysis, (d) taxane production analysis of cell suspension culture with and without exposure to ultrasound, (e) viability and growth analysis of the hazel (Corylus avellana) cells after subjecting to ultrasound, (f) analysis of the relation between duration of ultrasound sonication and production of iodine, (g) analysis of the relation between the spherical Bessel function and luminescence.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a block circuit diagram of spherical ultrasonic bioreactor for synthesizing taxol from hazel Corylus avellana, according to an embodiment herein.

FIG. 2 illustrates a photograph indicating a suspension culture in the spherical ultrasonic bioreactor, according to an embodiment herein.

FIG. 3 illustrates a flowchart indicating a method for synthesizing taxol from hazel Corylus avellana, according to one embodiment herein.

FIG. 4 illustrates the graph indicating an establishment of a fast growing cell line of hazel in weight in gms with respect to time in days in a method for synthesizing taxol from hazel Corylus avellana, according to one embodiment herein.

FIG. 5A, FIG. 5B and FIG. 5C jointly illustrate the graphs indicating the wavelength spectrum of the hazel cell extract obtained with a high performance liquid chromatography mass spectrometry (HPLC-MS) analysis of the hazel cell extract, in a method for synthesizing taxol from hazel Corylus avellana, according to one embodiment herein.

FIG. 6 illustrates the graph indicating the amount of hydrogen peroxide (H₂O₂) produced by the cell line of hazel under control conditions in spherical bioreactor with respect to ultrasonic exposure in minutes, in a method for synthesizing taxol from hazel Corylus avellana, according to one embodiment herein.

FIG. 7A illustrates the graphs indicating the DXR activity of the enzyme with respect to time during a production of taxol after exposure of hazel cells to ultrasound, in a method for synthesizing taxol from hazel Corylus avellana, according to one embodiment herein.

FIG. 7B illustrates the graphs indicating the relative intensity of DXR activity of the enzyme with respect to time during a production of taxol after exposure of hazel cells to ultrasound, in a method for synthesizing taxol from hazel Corylus avellana, according to one embodiment herein.

FIG. 7C illustrates the graphs indicating the PAL activity of the enzyme with respect to time during a production of taxol after exposure of hazel cells to ultrasound, in a method for synthesizing taxol from hazel Corylus avellana, according to one embodiment herein.

FIG. 8 illustrates the photograph indicating the gene expression patterns of the genes related to the production of taxol after exposure of hazel cells to ultrasound energy irradiation, in a method for synthesizing taxol from hazel Corylus avellana, according to one embodiment herein.

FIG. 9 illustrates the graph indicating a relationship between the amount of taxanes produced with respect to time in the suspension cell culture of hazel Corylus avellana with and without exposure to ultrasound, in a method for synthesizing taxol from hazel Corylus avellana, according to one embodiment herein.

FIG. 10 illustrates the graph indicating the net growth and the viability of suspension cell culture of hazel Corylus avellana after subjecting to ultrasound energy irradiation, in a method for synthesizing taxol from hazel Corylus avellana, according to one embodiment herein.

FIG. 11 illustrates the graph indicating the variation of iodine liberation with respect to time, in a method for synthesizing taxol from hazel Corylus avellana, according to one embodiment herein.

FIG. 12 illustrates the graph indicating the Bessel function in spherical bioreactor, used for synthesizing taxol from hazel Corylus avellana, according to one embodiment herein.

FIG. 13 illustrates the luminescence map of spherical bioreactor, used for synthesizing taxol from hazel Corylus avellana, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a piezoelectric, low intensity ultrasound bioreactor to investigate the effects of ultrasound on medicinal plant (Corylus avellana) tissue suspension culture for taxol production. The plant tissue culture for the production of taxol has many advantages. The useful phytochemical (taxol) is produced under controlled conditions independent of the external factors in which the plant/tree grows. The cultured cells and the medium are free from microbes and insects. The cells of any plants, tropical or alpine are easily multiplied to yield specific metabolites. The automated control of cell growth and regulation of metabolite processes reduces labor costs and increases productivity.

According to one embodiment herein, a spherical bioreactor system for synthesizing taxol from hazel (Corylus avellana) cell culture is provided. The system comprises a round bottomed flask, a plurality of piezoelectric transducers, an audio amplifier, a voltage controlled oscillator, integrator and a lock in amplifier. The piezoelectric transducers comprises a series of four pairs of piezoelectric crystals fixed together. The piezoelectric crystals are configured to induce a resonance in a circuit of system. The audio amplifier is configured to supply electrical power to the piezoelectric transducers. The audio amplifier is configured to amplify low power audio signals to a suitable level. The voltage controlled oscillator is configured to supply electrical power and generate ultra sound signals in the spherical bioreactor system. The voltage controlled oscillator transfers or supplies power to the audio amplifier. The integrator is configured to cumulate the input signal into an output signal. The integrator output signal is a time integral of the output signal. The lock in amplifier is configured to extract signals with a known carrier wave from a noisy environment. The oscillation frequency of the voltage controlled oscillator is controlled by an input voltage input. The input voltage estimates an instantaneous oscillation frequency. The voltage controlled oscillator modulates signals applied to control the input thereby causing frequency modulation or phase modulation. The system produces continuous ultrasound waves at preset frequency with different power output levels.

According to one embodiment herein, the round bottomed flask has a volume of 100 ml and the round bottomed flask is preferably made of glass.

According to one embodiment herein, the pluralities of piezoelectric transducers are two. The piezoelectric transducers have a length of 12.1 mm and the piezoelectric transducers have a diameter of 20 mm. The two piezoelectric transducers are mounted 180° degrees apart near a central portion of the round bottomed flask. The piezoelectric transducers are mounted on the round bottomed flask using a dry epoxy material.

According to one embodiment herein, the system produces ultrasound waves of 29 kHz with different levels of power output. The spatial peak intensity (Isp) of the system is in a range of 4-455 mW/cm³.

According to one embodiment herein, the method of synthesizing taxol in a spherical bioreactor from hazel (Corylusavellana) cell culture, comprises the following steps. The first step is obtaining the hazel (Corylusavellana) seeds tissue for cell line culture. A rapid growing cell line culture or callus culture is established from hazel seed tissue in a modified Murashige and Skoog medium (MS medium). A suspension culture is established from the cell line culture. The suspension culture is established from hazel seeds after growing the cell line culture or callus culture in modified the Murashige and Skoog medium (MS medium). The cells in a logarithmic growth phase is transferred along with culture media to a spherical ultrasonic bioreactor. The preliminary studies are conducted for estimating an ultrasound dosage and time for treating suspension culture with ultrasound signals. The cell line culture in the spherical ultrasonic bioreactor is treated or irradiated with the ultrasound of preset power levels for preset time periods. The cells are harvested after the ultrasound exposure. The harvested cells are washed and transferred to a fresh culture media for 1 week. The cells and culture media are analyzed or molecular and biochemical parameters. The cell suspension culture of the hazel (Corylus avellana) is analyzed for the molecular and biochemical parameters. The molecular and biochemical parameters are taxane production analysis. The cells are dried for extracting taxol. The intracellular and extracellular taxanes are extracted from the taxols.

According to one embodiment herein, a modified Murashige and Skoog (MS) medium comprises ammonium nitrate (NH₄NO₃) at a quantity of 1650 mgL⁻¹, monopotassium phosphate (KH₂PO₄) at a quantity of 170 mg L⁻¹, calcium chloride (CaCl₂) at a quantity of 332.02 mg L⁻¹, magnesium sulfate (MgSO₄) at a quantity of 180.54 mgL⁻¹, ferric-ethylenediamine-tetra-acetic acid (Fe-EDTA) at a quantity of 36.70 mgL⁻¹, boric acid (H₃BO₃) at a quantity of 6.20 mgL⁻¹, cupric sulphate pentahydrate (CuSO₄ 5H₂O) at a quantity of 0.025 mgL⁻¹, manganese(II) sulfate monohydrate (MnSO₄H₂O) at a quantity of 16.90 mg L⁻¹, sodium molybdate dehydrate (Na₂MoO₄ 2H₂O) at a quantity of 0.25 mg L⁻¹, zinc sulphate heptahydrate (ZnSO₄7H₂O) at a quantity of 8.60 mg L⁻¹, potassium iodide (KI) at a quantity of 0.83, Cobalt(II) chloride hexahydrate (CoCl₂6H₂O) at a quantity of 0.025 mg L⁻¹, and supplemented with 3% sucrose, and 1 mg L⁻¹ 4-dichlorophenoxyacetic acid and 0.5 mg L⁻¹ of benzyladenine. The pH of the modified Murashige and Skoog medium (MS medium) is 5.5.

According to one embodiment herein, the modified Murashige and Skoog medium (MS medium) for suspension culture of hazel seeds further comprises a naphthalene acetic acid (NAA) at a quantity of 3 mg L⁻¹ and a indole-3-acetic acid (IAA) at a quantity of 3 mg L⁻¹.

According to one embodiment herein, 50 subculture of the cell line a suspension culture is established, and the cells in suspension culture are incubated at 25° C. in dark with a shaking or agitation at 110 rpm on shaker incubator. The cells are sub-cultured every 7 days.

According to one embodiment herein, the cells are exposed to ultrasound energy of preset intensities for preset time periods. The preset intensity level of ultrasound energy for irradiating cell suspension culture is within a range of 4 to 455 mW/cm³. The preset time duration of ultrasound radiation for cell suspension culture is within a range of 2 to 60 min.

According to one embodiment herein, the cells are harvested, cultured and transferred to a fresh culture media after exposure to ultrasound energy. The fresh culture media is a modified Murashige and Skoog medium (MS medium) for cell growth and taxol production. The cells are cultured for 1 week in the modified Murashige and Skoog medium (MS medium).

According to one embodiment herein, the taxane production is analyzed by high performance liquid chromatography-mass spectrometry (HPLC-MS).

According to one embodiment herein, the step of analysing the cells and culture media for molecular and biochemical parameters comprises hydrogen peroxide (H₂O₂) production analysis, enzyme and enzyme activity analysis, an agarose gel electrophoresis analysis, a taxane production analysis of cell suspension culture with and without exposure to ultrasound energy, a viability and growth analysis of the hazel (Corylus avellana) cells after subjecting the cells to ultrasound irradiation, analysis of the relation between duration of ultrasound sonication and production of iodine and analysis of the relation between the spherical Bessel function and luminescence.

According to one embodiment herein, the enzymes analysed are 1-deoxy-D-xylulose-5-phosphate reducto-isomerase (DXR) and phenylalanine ammonia lyase (PAL).

According to one embodiment herein, the hydrogen peroxide (H₂O₂) is produced by the cell line of hazel under preset control conditions in the spherical bioreactor. The hydrogen peroxide (H₂O₂) is a reactive oxygen species. The hydrogen peroxide (H₂O₂) promotes a production of a taxol or taxanes. The hydrogen peroxide (H₂O₂) is a signaling molecule for stimulating a gene expression and promotes taxol production molecular pathway.

According to one embodiment herein, the hazel cells are exposed to ultrasound irradiation for a time period of 20 minutes to achieve a highest 1-deoxy-D-xylulose-5-phosphate reductoisomerase (DXR) activity in a harvesting time of 6 hours. The hazel cells are exposed to ultrasound irradiation for a time period of 20 minutes to achieve a highest phenylalanine ammonia lyase (PAL) activity in a harvesting time of 24 hours.

According to one embodiment herein, the highest taxol content is produced after exposing the hazel cell culture to ultrasound irradiation for 20 minute. A dry weight of the taxol content produced is 6 mg/Kg, when the hazel cell culture is exposed to ultrasound irradiation for 20 minute.

According to one embodiment herein, the exposure of hazel cells to ultrasound in the ultrasound bioreactor has no adverse effects on cell viability, cell growth and cellular membrane integrity.

According to one embodiment herein, the exposure of hazel cells to ultrasound has positive effects on biomass yields and increased taxanes biosynthesis.

According to one embodiment herein, the intracellular and extracellular taxanes are taxol, 10-deacetyl baccatin III and baccatin III.

According to one embodiment herein, the extraction of cell-associated intracellular and extracellular taxol comprises the following steps. The cells are collected from the culture medium. The collected cells are washed. The cells are dried for a predetermined period at room temperature. The predetermined drying time is 5 minutes. The dried cells are powdered at room temperature. The powdered cells are dissolved in 10 mL methanol to obtain a solution. The solution of powdered cell in methanol is ultrasonicated or irradiated with ultrasounds for 40 min to obtain a homogenate. The homogenate is filtered. The filtrate is air dried and re-dissolved in a mixture of methylene chloride and water. The methylene chloride and water are mixed in a ratio of 1:1. The dissolved filtrate is centrifuged at 5000 rpm to obtain a supernatant and a pellet. The supernatant is eluted to obtain a methylene chloride phase. The methylene chloride phase is collected. The methylene chloride phase is air-dried and re-dissolved in a 250 mL methanol. The dissolved methylene chloride phase in methanol is filtered with a 0.45-mm syringe filter. The filtrate is subjected to a high-performance liquid chromatography (HPLC) analysis. The HPLC system is equipped with a C-18 column. The extracellular taxol (in the medium) with methylene chloride (1:1) is extracted in a separating funnel. The taxol is eluted at a flow rate of 1 mL min-1 methanol and water (45:55, v/v). The taxol is detected at 227 nm using an ultraviolet detector. The quantity of taxol produced is estimated by comparison of a retention time and peak area with that of a genuine standard.

According to one embodiment herein, taxol (generic name, paclitaxel) is a well known anticancer drug. Taxol is a diterpene alkaloid which is usually isolated from the bark of the yew tree (Taxus species). Despite the use of taxol as a cure for cancer, the isolation of taxol from the bark of the Pacific yew trees is time consuming, expensive and ineffective. The tree is slow growing and two to four of it is required to obtain enough taxol to treat one patient. Another tree hazel (Corylus avellana) is widely available and hazel cells grow faster in vivo and are easily cultivated in vitro than yew.

According to one embodiment herein, ultrasound has been widely applied in medicine and biology for diagnosis and therapy during many decades. The biological effects of ultrasound have received considerable attentions. High intensity ultrasound is well known to be destructive to biological materials, disrupting the cell membranes and deactivating biological molecules such as enzymes and DNA. Low-intensity ultrasound on the other hand has shown a range of sub-lethal biological effects that are of potential significance in biotechnology. Series of experiments are conducted using ultrasonic bath with different power densities, different periods and repetitions of exposure to ultrasound on hazel cells.

Low-intensity ultrasound is known as a physical elicitor. It has a various effects on biological pathways in the living cells including the increase of reactive oxygen species (ROS) production. The mechanical stress of ultrasound on cells suspended in liquid medium arises from two hydrodynamic events. The hydrodynamic events are acoustic cavitation and cavitation induced microstreaming. These hydrodynamic events results in shear stress, subsequently producing ROS. The ROS affects the plant cells, such as causing damage to cell membranes, essential macromolecules such as proteins, DNA, and lipids. The ROS also function as signal molecule to trigger a series of cellular responses from expression of genes to produce taxanes. In the spherical ultrasound bioreactor, due to specificity of growth phase, exposure duration, and ultrasound dosage a definite amount of ROS are produced. The level of ROS produced in ultrasound bioreactor does not destruct the plant cells but is efficient enough to promote taxane production pathway.

According to one embodiment herein, a piezoelectric type, low intensity ultrasound generating apparatus (a spherical ultrasonic bioreactor) is designed in order to investigating the effects of ultrasounds on tissue culture of medicinal plant. Particularly the effect of ultrasound on suspension culture of hazel cells and the analysis is done for possibility of increase in taxol production.

According to one embodiment herein, the spherical ultrasound bioreactor is composed of two piezoelectric transducers (12.1 and 20 mm) in diameter and length respectively). The transducers are mounted 180° apart as near to the equator of the bottom of 100 ml flask. The transducers are mounted using a quick dry epoxy attached to both sides of bioreactor. Each transducer is composed of a series of 4 two piezo-ceramic crystals fixed together so that the circuit resonates and maximum power is delivered to flask. Power is supplied through a voltage controlled oscillator (Juya Electronic Co. Iran). The resonance frequency of the flask is determined by searching for peaks in the amplitude of the sound field in the reactor by tracking the phase difference between voltage and current of the transducer. The device produces continuous ultrasound waves at 29 kHz with different levels of power output. Acoustic calibration for the power and intensity of the device is carried out in degassed water in the tank, using a radiation force balance (Shrewbury Medical Co. UK) and the hydrophone method in the cubic chamber (PA124, Precision Acoustics Ltd Dorchester Dorset UK). The calibration range is 10 kHz-20 MHz, with a 25 mm sensor diameter. The measurements are carried out when the distance between head of hydrophone and the device is 1 cm. The real frequency and actual spatial peak intensities (Isp) of this ultrasonic unit are 29 KHz and 4-455 mW/cm³ respectively, fundamental frequency based on calibration certification test results. To find the appropriate exposure dosage a set of preliminary studies are conducted using different durations of 2 to 60 minutes and power intensities of 4-455 mW/cm³.

According to one embodiment herein, a fast growing cell line for hazel Corylus avellana is established. The cell line and cell culture serve as renewable source of taxanes. In the bioreactor the controlled production of taxol and other anti-cancer drugs is possible. The growth of the suspension culture of hazel cells is achieved under controlled conditions independent of climatic changes or soil changes, free from microbial infection and insects. The cells are multiplied to yield specific metabolites to gather with reduced labor costs and improved productivity. The taxanes are extracted from cultured plant tissue culture.

According to one embodiment herein, hazel cells are grown in suspension cultures in shake-flasks with the specific culture conditions and specific culture medium formulation. The growth of the cells is plotted against time and the appropriate time for application of ultrasound is decided. The cells are exposed to definite power density of ultrasound just for a short period (found by other sets of experiments). The cells are then transferred back to their batches in order to continue to growth and Taxol production. The ultrasound in bioreactor is applied in order to stimulate the cells for Taxol production and not for monitoring the cell status. This ultrasound bioreactor system is not destructive to the cells and they can be sub-cultured and their produced drugs be harvested from medium continuously.

According to one embodiment herein, a rapidly growing cell line is induced from the seeds of Iranian cultivar of Corylus avellana, which is cultivated in northern Iran. The callus is established in a modified Murashige and Skoog medium (MS medium) without glycine and supplemented with 3 mg NAA/L, 3 mg ISS/L and 1 mg kinetin/L. Where 1-naphthalene acetic acid is NAA and indole-3-acetic acid is IAA respectively. The suspension culture is established by transferring 1 gram callus in 30 ml culture medium. The callus in culture medium is incubated at 25° C. in a dark environment on a rotary shaker with 110-120 rpm. The growth curve of the cells is plotted in order to find appropriate times for manipulation of the cells. The suspension is renewed every seven days. Further various strategies are employed to increase the production of taxol in hazel cell cultures, i.e. addition of different precursor, elicitation with different chemicals and physical elicitors (including magnetic fields and different types of ultrasounds) or combination of these strategies.

According to one embodiment herein, ultrasound is widely applied in medicine and biology for diagnosis and therapy during many decades. The result illustrates that short exposure of hazel cells to ultrasound enhances both extracellular and cell-associated taxol contents. The extracellular yield of taxol increases. This increase is attributed to ultrasound-induced temporary increase of membrane permeability that facilitated the release of paclitaxel into the medium. Further results illustrate that exposure of hazel cells to ultrasound in this bioreactor has no adverse effects on cell viability, cell growth and membrane integrity but also excreted beneficial effects on biomass yields and increase in taxol yield. The system has the potential to produce appropriate amounts of signaling molecules thereby regulate activities of stress related enzymes and expression of their genes example: catalase. The ultrasound bioreactor system also enhances the expression of phenylalanine ammonia lyase (PAL), a key enzyme involved in signaling transduction pathways resulted in paclitaxel production and 1-deoxy-D-xylulose-5-phosphatereductoisomerase (DXR), a key enzyme of paclitaxel production pathway.

According to one embodiment herein, a piezoelectric type, low intensity ultrasound generating apparatus (spherical ultrasonic bioreactor) is designed in order to investigate the effects of ultrasounds on cell suspension-culture of hazel cells and the possibility of increase of taxol production by them.

The spherical ultrasonic bioreactor is composed of two piezoelectric transducers (12.1 and 20 mm in diameter and length, respectively). The piezoelectric transducers are mounted 180 degrees apart as near to the equator of a round bottom 100 mL flask. The piezoelectric transducers are mounted on both sides of bioreactor using a quick dry epoxy. Each transducer is composed of a series of 4 two piezo-ceramic crystals fixed together so that resonate the circuit, thereby delivering maximum power to the flask. The electric power is supplied through a voltage-controlled oscillator. The resonant frequency of the flask is determined by analyzing the peaks in the amplitude of the sound field of the reactor by tracking the phase difference between voltage and current of the transducer. The spherical ultrasonic bioreactor produces continuous ultrasound waves at 29 kHz with different levels of power output. Acoustic calibration for the power and intensity of the device is carried out in degassed water in a tank, using a radiation force balance (Shrewsbury Medical Co, UK) and by the hydrophone method in the cubic chamber (PA124, Precision Acoustics Ltd, Dorchester, Dorset, UK, calibration range: 10 kHz-20 MHz, with a 25 mm sensor diameter). The power and intensity measurements are carried out when the distance between head of hydrophone and the device is 1 cm. The real frequency and actual spatial peak intensities (Isp) of this ultrasonic unit are, 29 kHz and 4-455 mW/cm³ respectively, based on fundamental frequency calibration certification test results. To find the appropriate exposure dosage, a set of preliminary studies are conducted using different durations ranging 2 to 60 min) and power intensities ranging 4-455 mW/cm3).

According to one embodiment herein, a rapid growing cell line is established from the seeds of Iranian cultivar of hazel (Corylus avellana), in a modified Murashige and Skoog (MS) medium comprising (mg L−1): ammonium nitrate (NH₄NO₃) 1650, monopotassium phosphate (KH₂PO₄) 170, calcium chloride (CaCl₂) 332.02, magnesium sulfate (MgSO₄) 180.54, ferric-ethylenediamine-tetra-acetic acid (Fe-EDTA) 36.70, boric acid (H₃BO₃) 6.20, cupric sulfate pentahydrate (CuSO₄ 5H₂O) 0.025, manganese(II) sulfate monohydrate (MnSO₄H₂O) 16.90, Sodium molybdate dehydrate (Na₂MoO₄2H₂O) 0.25, zinc sulfate heptahydrate (ZnSO₄ 7H₂O) 8.60, potassium iodide (KI) 0.83, Cobalt(II) chloride hexahydrate (CoCl₂6H₂O) 0.025, and supplemented with 3% sucrose, and 1 mg L−1 2,4-dichlorophenoxyacetic acid and 0.5 mg L−1 benzyladenine at pH 5.5.

After 50 subcultures of the cell line, suspension cultures are established in a modified medium basically similar to the aforesaid but supplemented with 3 mg L−1-naphthalene acetic acid (NAA) and 3 mg L-lindole-3-acetic acid (IAA). The cells are incubated at 25° C. in dark with shaking at 110 rpm on shaker incubator. The cells are sub-cultured every 7 days. The cells (in their logarithmic growth phase) together with their media are transferred in a spherical ultrasonic bioreactor and are exposed to ultrasound of definite power densities and periods. After ultrasound exposure, the cells are cultures, harvested, thoroughly washed, and transferred back to fresh media in order to allow cells to grow and complete taxol production for further 1 week. The extracted cells are also thoroughly washed and dried for the taxol extraction.

For extraction of cell-associated (intracellular) Taxol, the dried cells are powdered for 5 min at room temperature. The powder of cells is dissolved in 10 mL methanol and ultrasonicated for 40 min to obtain a homogenate. The homogenate is filtered. The filtrate is air dried and re-dissolved in a mixture of methylene chloride and water (1:1). The dissolved filterate is centrifuged at 5000 rpm. After centrifugation the methylene chloride phase is collected, air-dried and re-dissolved in 250 mL methanol and filtered with a 0.45-mm syringe filter before high-performance liquid chromatography (HPLC) analysis. Furthermore, extracellular Taxol (in the medium) is extracted with methylene chloride (1:1) in a separating funnel. The methylene chloride phase is collected, air dried, re-dissolved in 250 mL methanol and filtered with a 0.45-mm syringe filter for high-performance liquid chromatography (HPLC) analysis. The HPLC system (Knauer, Berlin, Germany) is equipped with a C-18 column (Perfectsil Target ODS3, 5 mm, 250×4.6 mm, MZ-Analysentechnik, Mainz, Germany). The taxol is eluted at a flow rate of 1 mL min⁻¹ methanol and water (45:55, v/v) and is detected at 227 nm using an ultraviolet detector (PDA, Berlin, Germany). The quantification of taxol is accomplished by comparison of retention time and peak area with genuine standard (Sigma, St. Louis, Mo., USA).

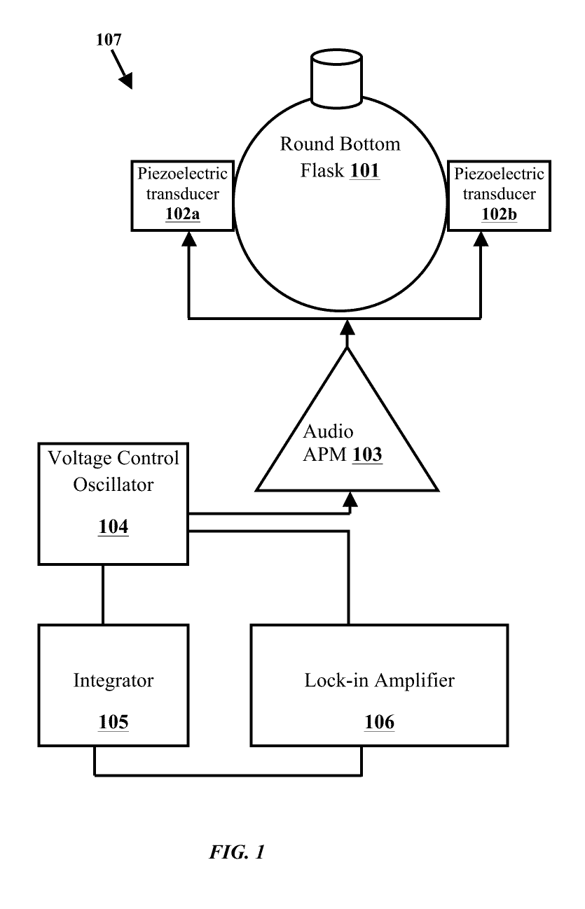

FIG. 1 illustrates a schematic circuit diagram of spherical ultrasonic bioreactor, according to an embodiment herein. The piezoelectric type, low intensity ultrasound generating apparatus (spherical ultrasonic bioreactor) is designed to investigate the effects of ultrasounds on suspension-cultured hazel cells and the effect in increase of Taxol production.

The spherical ultrasonic bioreactor 107 comprises of a round bottom 100 mL flask 101, two piezoelectric transducers 102a and 102b (12.1 and 20 mm in diameter and length, respectively), audio amplifier (audio AMP) 103, voltage control oscillator 104, integrator 105 and lock-in amplifier 106. The piezoelectric transducers 102a and 102b are mounted 180 degrees apart as near to the equator of a round bottom 100 mL flask 101 using quick dry epoxy attached to both sides of bioreactor. Each transducer comprises of a series of four pairs of piezo-ceramic crystals fixed together so that the circuit resonate, thereby delivering maximum power to the flask. Power/signals are supplied through a voltage-controlled oscillator 104. The audio amplifier (audio AMP) 103 supplies power to the piezoelectric transducers 102a and 102b. The function of audio AMP 103 is to amplify low power audio signals to a level suitable for driving loudspeakers. The audio power amplifier (audio AMP) 103 is connected to the voltage control oscillator 104. The voltage control oscillator's oscillation frequency is controlled by voltage input. The applied input voltage determines the instantaneous oscillation frequency. Consequently modulating signals applied to control input cause frequency modulation (FM) or phase modulation (PM). The voltage control oscillator 104, integrator 105 and lock in amplifier 106 are interconnected. The integrator 105 is the component whose output signal is the time integral of its input signal. The integrator 105 continuous analog of a counter, cumulating the input into an output. The lock in amplifier 106 is a type of amplifier that extracts a signal with a known carrier wave from an extremely noisy environment. The resonant frequency of the round bottom flask is determined by searching for peaks in the amplitude of the sound field in the reactor by tracking the phase difference between voltage and current of the transducer. The device produced continuous ultrasound waves at 29 kHz with different levels of power output.

Acoustic calibration for the power and intensity of the device is carried out in degassed water in the tank, using a radiation force balance (Shrewsbury Medical Co, UK) and the hydrophone method in the cubic chamber (PA124, Precision Acoustics Ltd, Dorchester, Dorset, UK, calibration range: 10 kHz-20 MHz, with a 25 mm sensor diameter. The measurements are carried out when the distance between head of hydrophone and the device is 1 cm. The real frequency and actual spatial peak intensities (Isp) of the ultrasonic unit are, 29 kHz and 4-455 mW/cm³ respectively, based on fundamental frequency calibration certification test results. To find the appropriate of exposure dosage, a set of preliminary studies are conducted using different durations (ranging 2 to 60 min) and power intensities (ranging 4-455 mW/cm³).

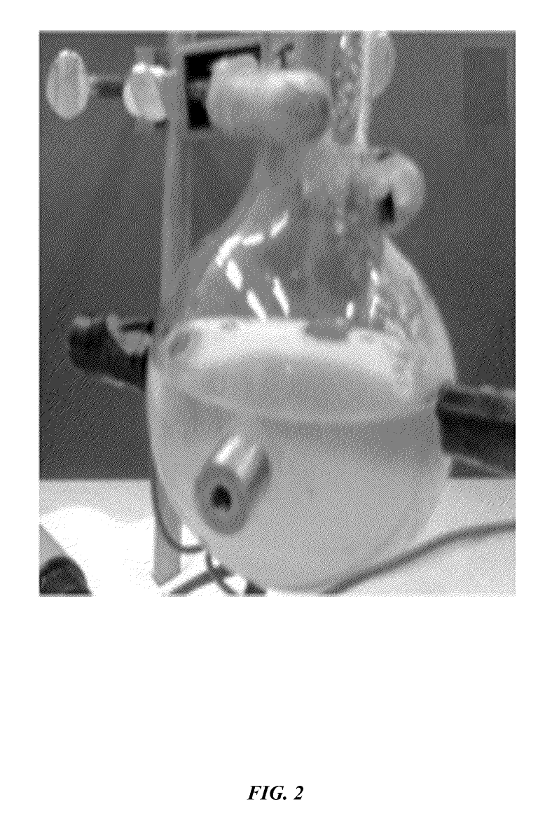

FIG. 2 illustrates a photograph showing the spherical ultrasonic bioreactor with suspension culture, according to an embodiment herein. The FIG. 2 shows the suspension culture of the hazel (Corylus avellana).

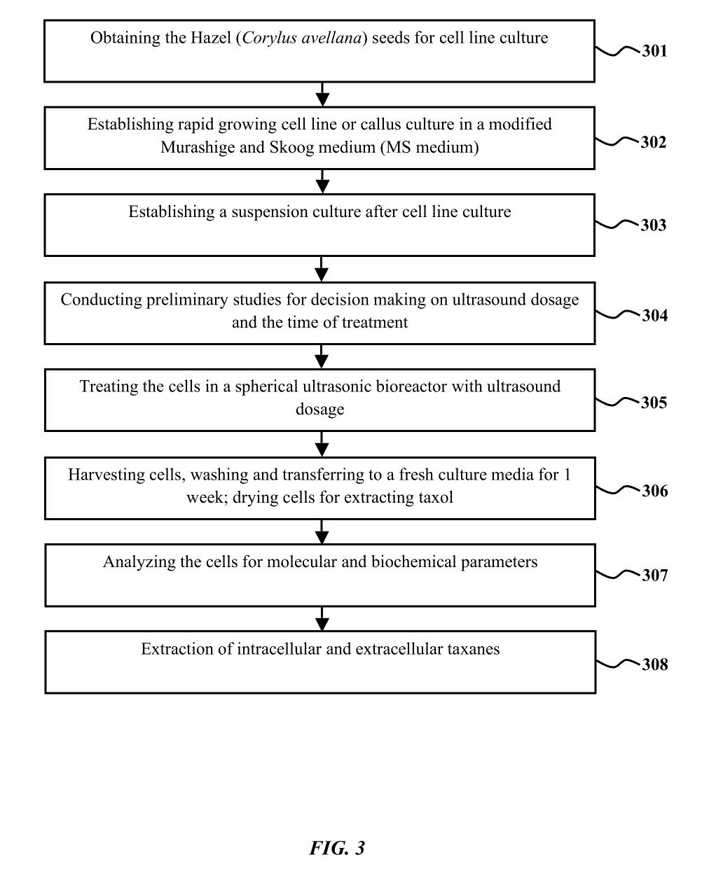

FIG. 3 illustrates a flowchart indicating a method for producing taxol from hazel Corylus avellana, according to one embodiment herein. With respect to FIG. 3, the first step is obtaining the Hazel (Corylus avellana) seeds for cell line culture (301). The next step is establishing rapid growing cell line or callus culture in a modified Murashige and Skoog medium (MS medium) (302). Further establishing a suspension culture after cell line culture (303). The next step is conducting preliminary studies for decision making on ultrasound dosage and the time of treatment (304). Further the cells are treated in a spherical ultrasonic bioreactor with ultrasound dosage (305). After treating the cells, next step is harvesting the cells, washing and transferring the cells to a fresh culture media for 1 week, the cells are also dried for extracting taxol (306). The next step includes analyzing the cells for molecular and biochemical parameters (307). The harvested cells are subjected to extraction of intracellular and extracellular taxanes (308).

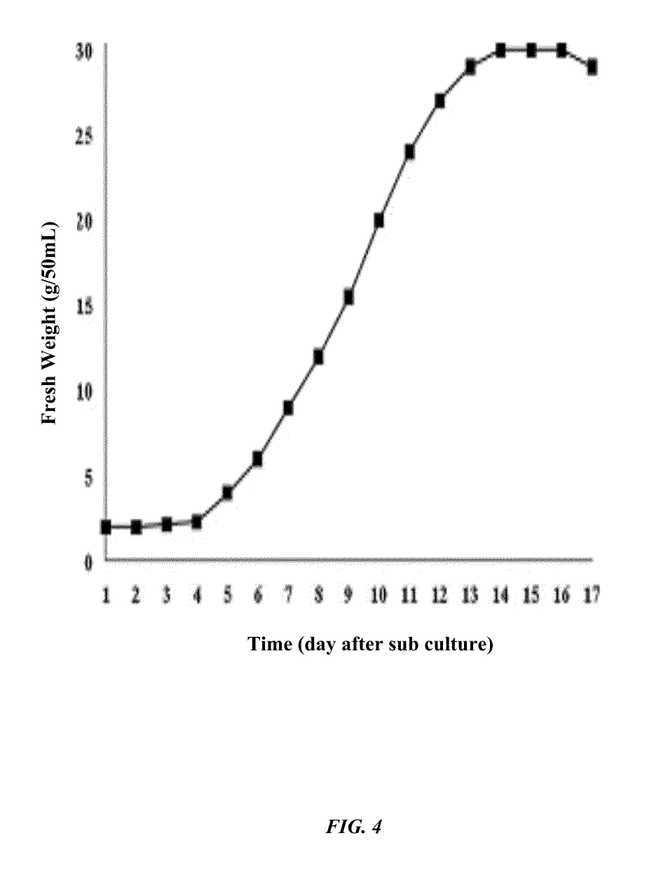

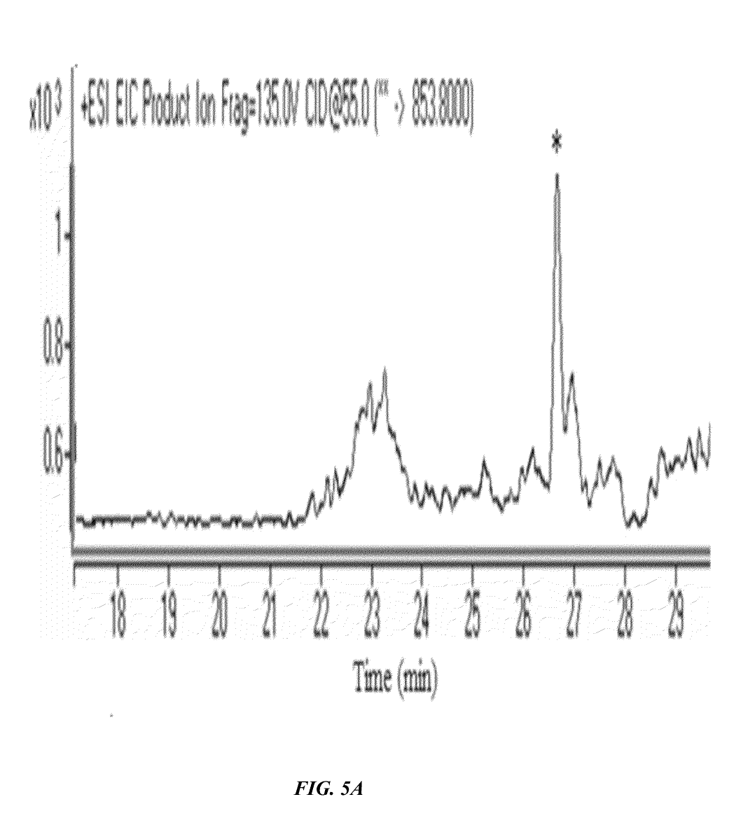

FIG. 4 illustrates the graph indicating the establishment of a fast growing cell line of hazel, according to one embodiment herein. FIG. 4 illustrates the growth curve of rapid growth in hazel cell line. The graph also shows that during two weeks of culture cell biomass increases up to 15 folds of its initial weight.

According to one embodiment herein, the cell suspension culture of the hazel (Corylus avellana) are analyzed for the following: (a) analysis of taxane production by high performance liquid chromatography-mass spectrometry (HPLC-MS), (b) hydrogen peroxide (H₂O₂) production analysis, (c) Enzyme and enzyme activity analysis (1-deoxy-D-xylulose-5-phosphate reductoisomerase (DXR) and phenylalanine ammonia lyase (PAL) and agarose gel electrophoresis analysis, (d) taxane production analysis of cell suspension culture with and without exposure to ultrasound, (e) viability and growth analysis of the hazel (Corylus avellana) cells after subjecting to ultrasound, (f) analysis of the relation between duration of ultrasound sonication and production of iodine, (g) analysis of the relation between the spherical Bessel function and luminescence.

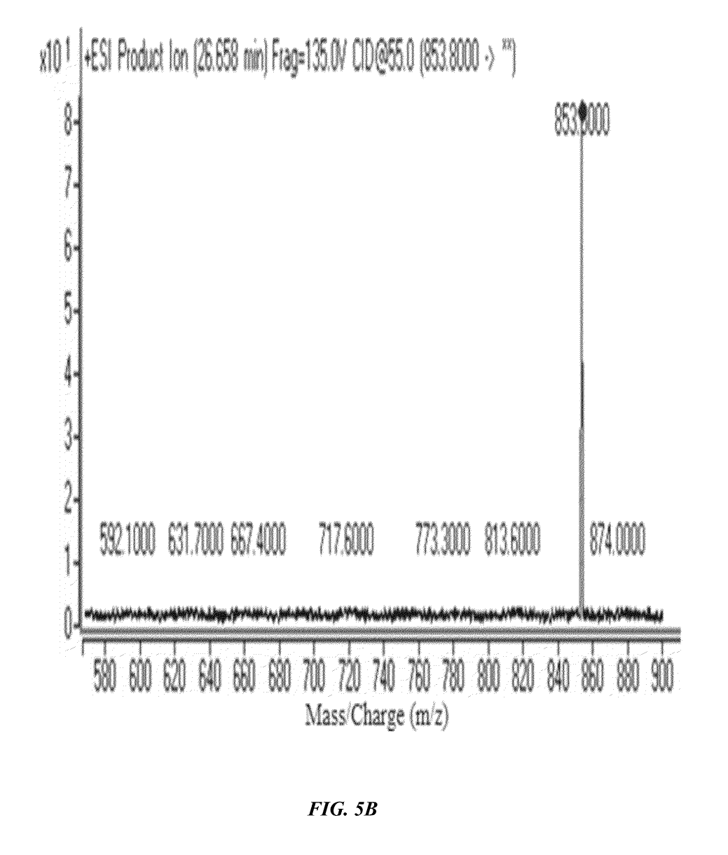

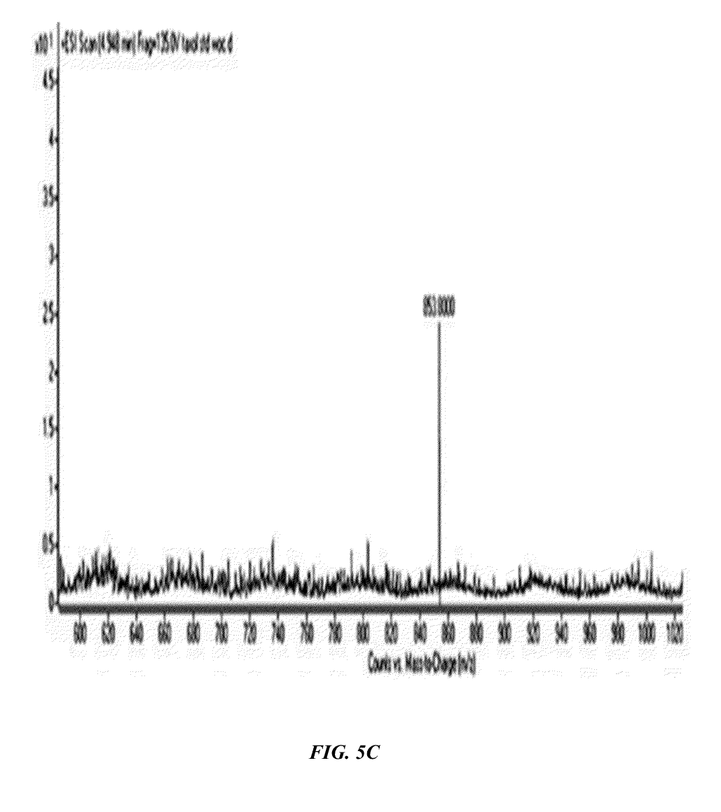

FIG. 5A-5C illustrates the graphs indicating the results of high performance liquid chromatography mass spectrometry (HPLC-MS) analysis of the hazel cell extract, according to one embodiment herein. FIG. 5A illustrates the amount of taxol in the hazel cell extract sample. The asterisk shows the peak and the amount of taxol. FIG. 5B illustrate the ion mass spectrum of the taxol in the hazel cell extract sample. The FIG. 5C illustrate the mass spectrum of the standard taxol.

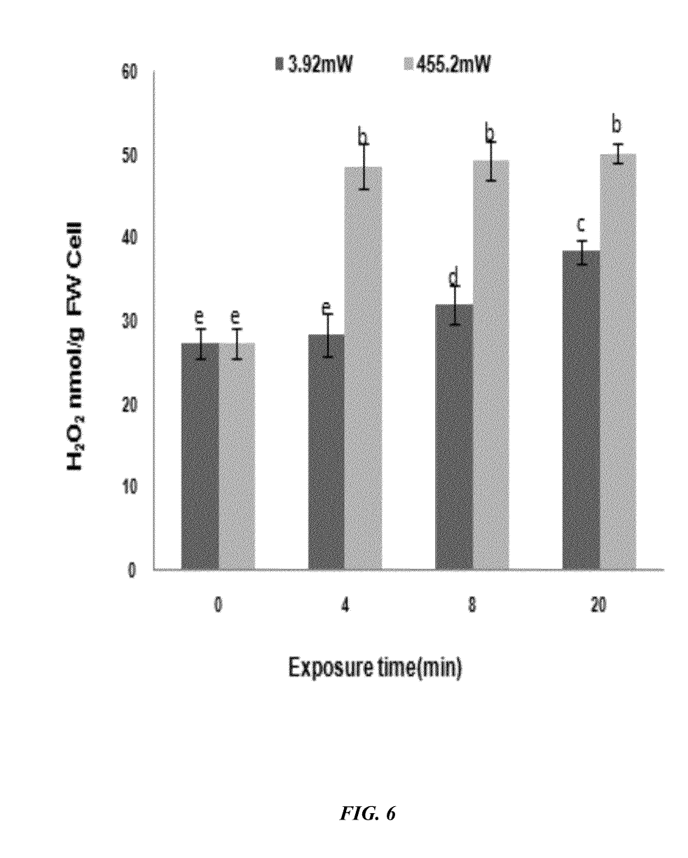

FIG. 6 illustrates the graph indicating the amount of hydrogen peroxide (H₂O₂) produced by the cell line of hazel under control conditions in spherical bioreactor, according to one embodiment herein. The data in the graph is presented as mean±SD with n=3. Further the bars in the graph with different letters are significantly different at p<0.05 according to LSD test. The graph illustrates that there is significant production of hydrogen peroxide (H₂O₂). The production of hydrogen peroxide (H₂O₂) promotes the production of enzymes related to taxol production. The FIG. 6 illustrates that the hazel cells produce reactive oxygen species in the spherical bioreactor. Specifically the hazel cell culture produces the hydrogen peroxide (H₂O₂).

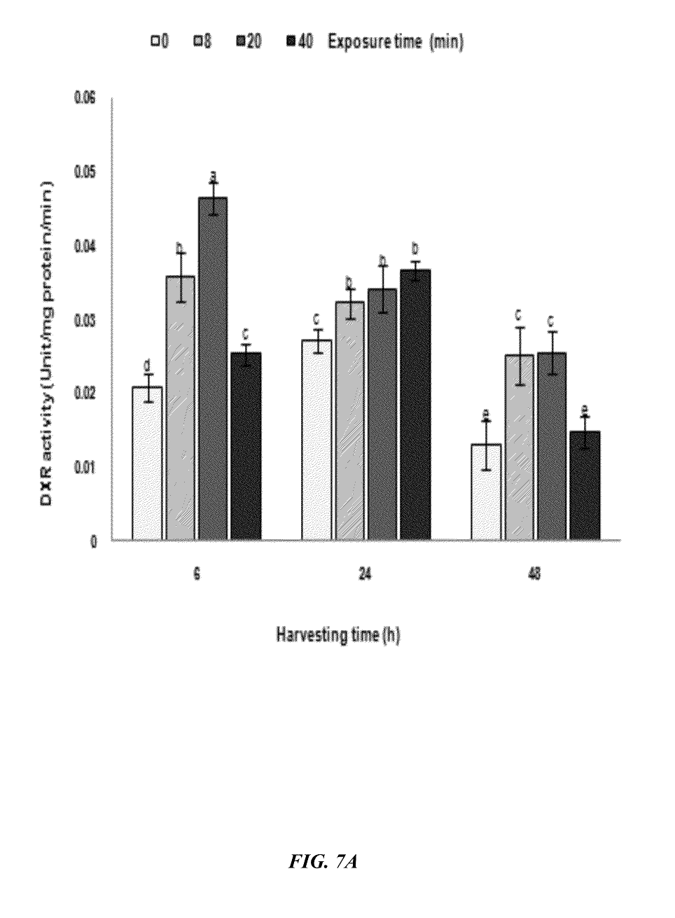

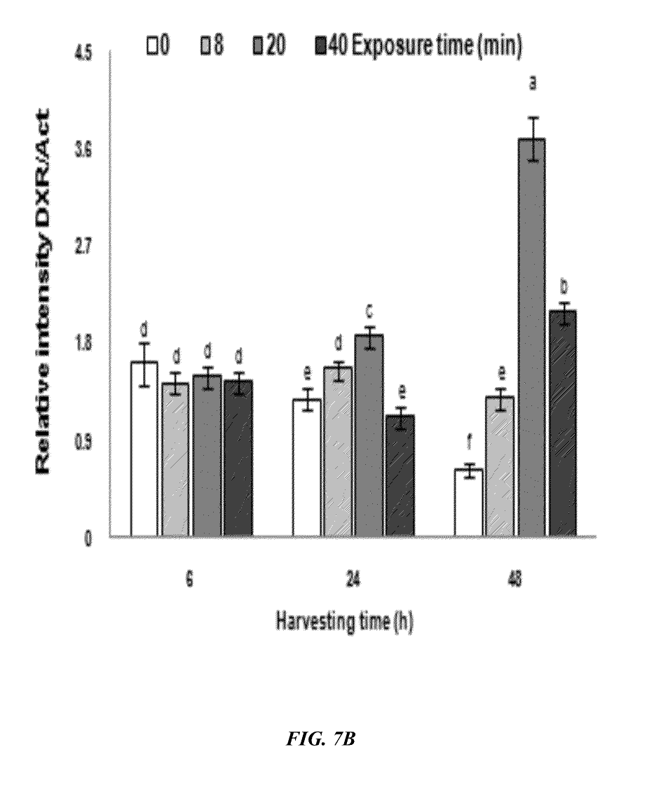

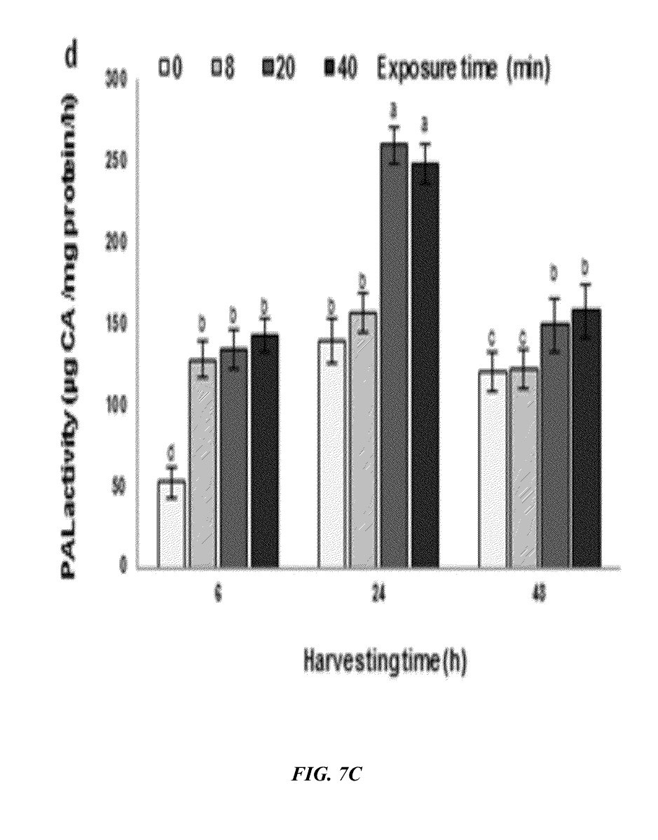

FIG. 7A-7C illustrates the graphs indicating the stimulation of the enzyme activity related to the production of taxol after exposure of hazel cells to ultrasound, according to one embodiment herein. FIG. 7A illustrate the activity of the enzyme 1-deoxy-D-xylulose-5-phosphate reductoisomerase (DXR). FIG. 7A further illustrates that exposing hazel cells to ultrasound for a time period of 20 minutes shows the highest DXR activity in the harvesting time of 6 hours. FIG. 7B illustrate the relative expression profile of DXR normalized with actin as an internal control. FIG. 7C illustrate the activity of the phenylalanine ammonia-lyase (PAL). The FIG. 7C further illustrates that exposing hazel cells to ultrasound for a time period of 20 minutes shows the highest PAL activity in the harvesting time of 24 hours. The data for FIG. 7A-FIG. 7C is presented as mean±SD with n=3. The bars in the graphs with different letters in each graph are significantly different at p<0.05 according to LSD test.

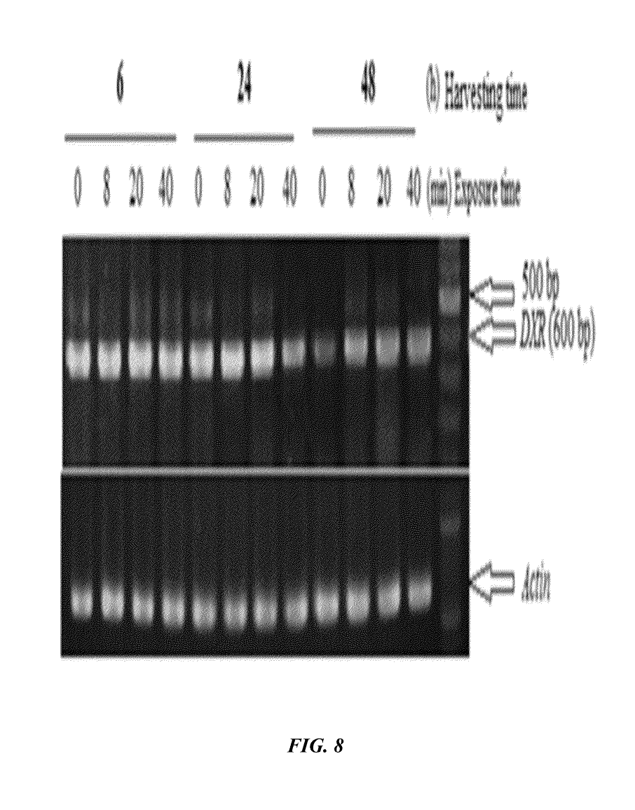

FIG. 8 illustrates the photograph indicating the expression patterns of the genes related to the production of taxol after exposure of hazel cells to ultrasound, according to one embodiment herein. FIG. 8 illustrates the RT-PCR product analysis of 1-deoxy-D-xylulose-5-phosphate reductoisomerase (DXR) gene and phenylalanine ammonia-lyase (PAL) gene.

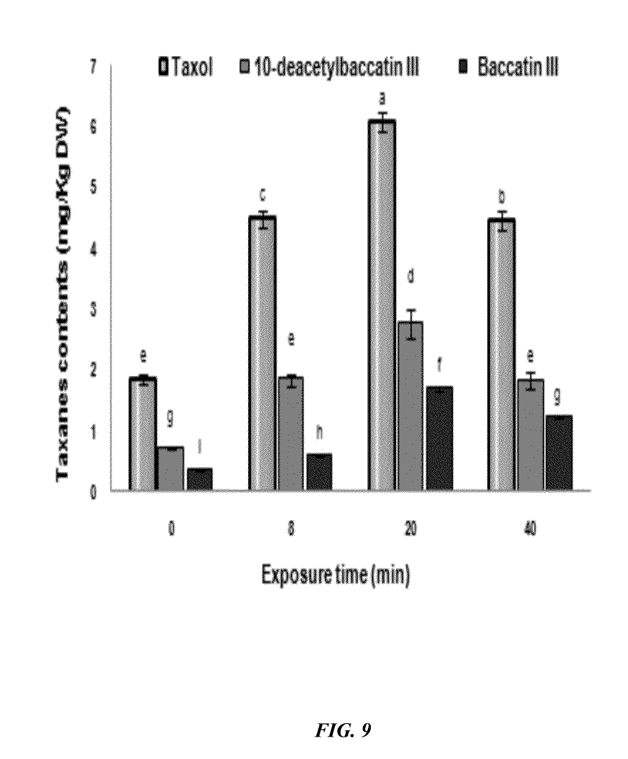

FIG. 9 illustrates the graph indicating the amount of taxanes produced the suspension cell culture of hazel Corylus avellana with and without exposure to ultrasound, according to one embodiment herein. FIG. 9 illustrate highest taxol content/production is achieved when the hazel cell culture is exposed to ultrasound for 20 minute. The taxol content is 6 mg/Kg dry weight when the exposure to ultrasound is for 20 minute. FIG. 9 also illustrates the production of 10-deacetylbaccatin III and baccatin III. The data for FIG. 9 is presented as mean±SD with n=3. The bars in the graphs with different letters in each graph are significantly different at p<0.05 according to LSD test.

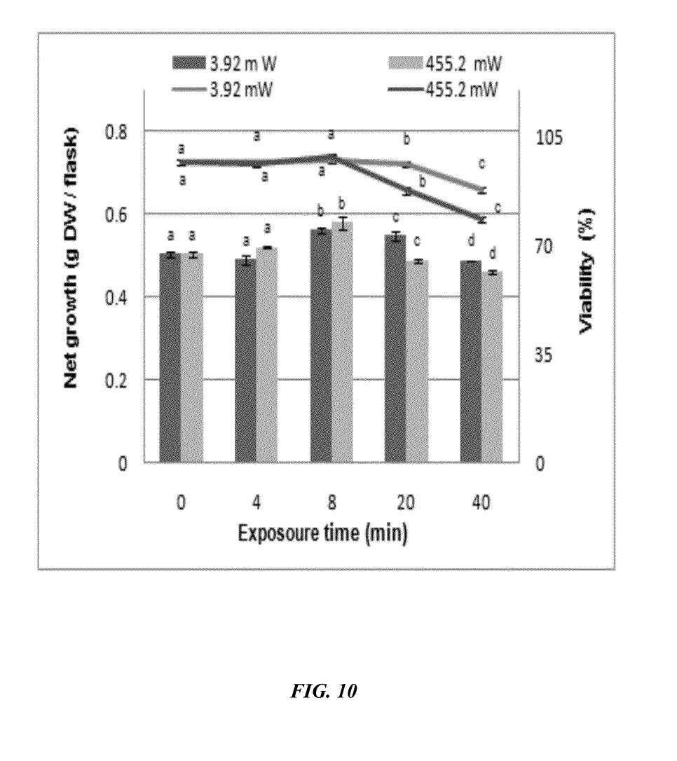

FIG. 10 illustrates the graph indicating the viability and growth of suspension cell culture of hazel Corylus avellana after subjecting to ultrasound, according to one embodiment herein. The FIG. 10 illustrates that the exposure of hazel cells to ultrasound in the ultrasound bioreactor, has no adverse effects on cell viability, growth and membrane integrity. FIG. 10 also illustrates that the exposure of hazel cells to ultrasound has positive effects on biomass yields and increased taxanes biosynthesis. Six day old hazel Corylus avellana cell culture is exposed to ultrasound at different power densities of 4 and 455 mW for 4-40 minutes. The cells dry weight (DW) and viability are measured after 7 days. FIG. 10 illustrates that the exposure of hazel cells to ultrasound has no adverse effect on the viability and growth of suspension cultures hazel cells. The data for FIG. 10 is presented as mean±SD with n=3. The bars in the graphs with different letters in each graph are significantly different at p<0.05 according to LSD test.

According to one embodiment herein, by application of ultrasound the bubbles collapse through cavitation and reactive oxygen species (ROS) are produced. This phenomenon is usually assumed to be occurred when ultrasound of high intensity is applied. The low intensity ultrasound in spherical bioreactor is also capable to produce sufficient amount of ROS, thereby promote taxanes production pathway. The occurrence of hydroxyl (.OH) radicals in spherical bioreactor is monitored by potassium iodide (KI) dosimetry as well as luminescence of Luminol.

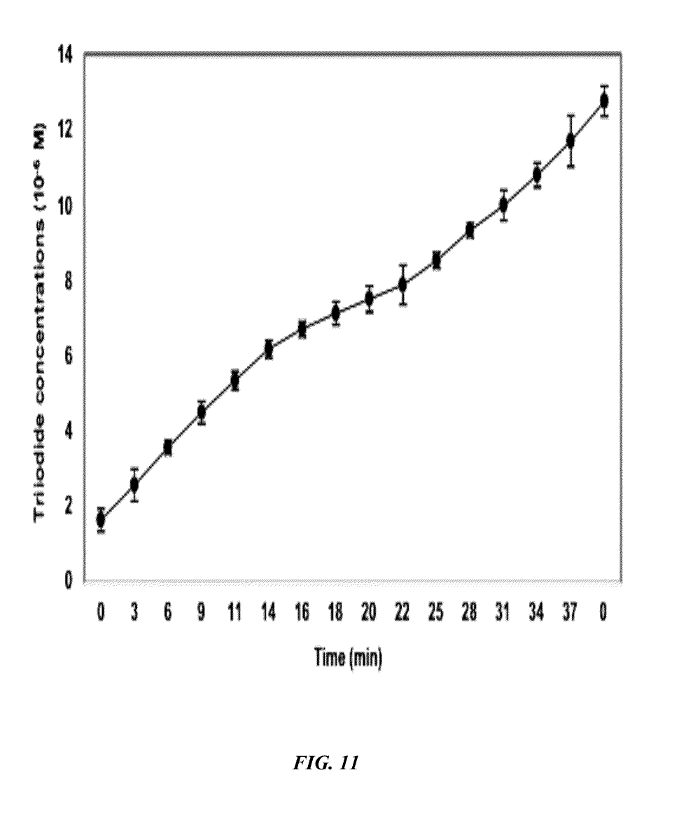

For potassium iodide (KI) dosimetry a solution comprising iodine and potassium iodide is prepared and diluted gradually. This solution is used to prepare a calibration curve of iodine in KI versus absorbance measured at 350 nm, with KI solution as a blank. FIG. 11 illustrates the graph indicating the variation of iodine liberation with time, according to one embodiment herein. FIG. 11 illustrates a plot of absorbance versus different KI concentrations yielded a straight line (R2=0.998) of positive slope for concentration from 0.005 to 0.100 M. The absorbance is proportional to I₃-ions formation. The inertial cavitation activity is estimated by the absorbance of sonication samples. The amounts of .OH radicals produced by ultrasound is defined from the rate and amount of the iodine liberation. Decomposition of KI is used as a factor indicating the intensity of cavitation and therefore leads to establish of mathematical relationships between the overall macroscopic rates and the intensity of cavitation. The sonication is performed for 0-40 min at 5 min intervals. FIG. 11 illustrates the relationship between duration of sonication and production of I₃. As shown, the concentration of I₃-increase along with increasing sonication period, in a linear mode.

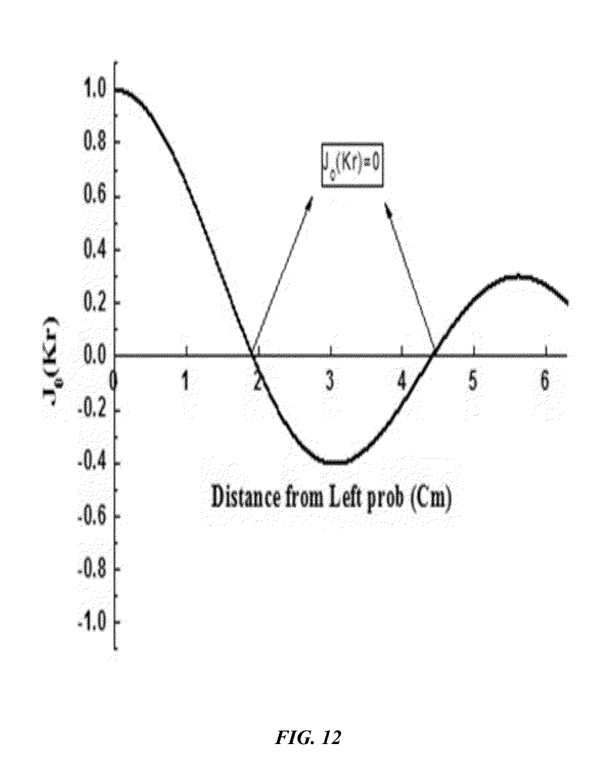

FIG. 12 illustrates the graph indicating the Bessel function in spherical bioreactor, according to one embodiment herein. The source of ultrasonic irradiation (two piezoelectric transducers) are mounted 180 degrees apart as near to the equator of a round bottom 100 mL flask as possible. The sound wave equation is a Spherical Bessel equation JO (Kr) where K is wave number and r is the distance from source of ultrasonic irradiation. For (Kr) of 2.40 and 5.52, JO (Kr) will be zero. For a frequency of ca. 30 kHz, and sound velocity in water at 30° C., the nodes will be formed at 1.91 and 4.39 cm of ultrasonic source.

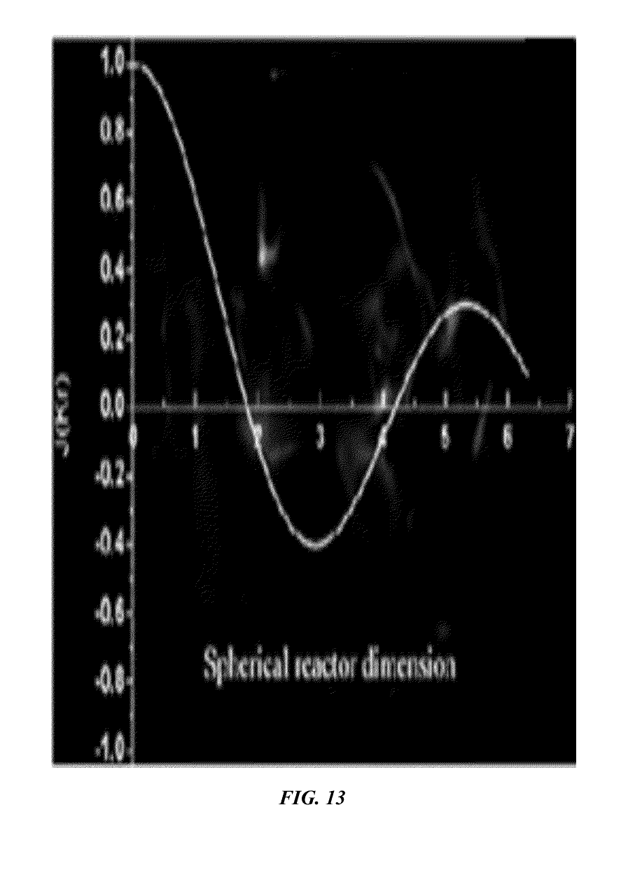

FIG. 13 illustrates the luminescence map of spherical bioreactor, according to one embodiment herein. Given the bioreactor diameter (6.3 cm) the two nodes will overlap. When the two piezoelectric transducers are on, then a standing wave will be formed. Hence the number of bubbles collapsing (i.e., the number of .OH radicals) are maximum in the location of nodes. Subsequently, the maximum intensity of luminescence of luminol (resulted from its interaction with .OH radicals) is observed in the nodes location.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A spherical bioreactor system for synthesizing taxol from hazel (Corylus avellana) cell culture comprises:
a round bottomed flask;
a plurality of piezoelectric transducers, and wherein the piezoelectric transducers comprises of a series of four pairs of piezoelectric crystals fixed together, and wherein the piezoelectric crystals are configured to induce a resonance in a circuit of system;
an audio amplifier, and wherein the audio amplifier is configured to supply electrical power to the piezoelectric transducers, and wherein the audio amplifier is configured to amplify low power audio signals to a suitable level;
a voltage controlled oscillator, and wherein the voltage controlled oscillator is configured to supply electrical power and generate ultra sound signals in the spherical bioreactor system and wherein the voltage controlled oscillator transfers or supplies power to the audio amplifier;
an integrator and wherein the integrator is configured to cumulate the input signal into an output signal, and wherein the integrator output signal is a time integral of the output signal; and
a lock in amplifier, wherein the lock in amplifier is configured to extract signals with a known carrier wave from a noisy environment;
wherein an oscillation frequency of the voltage controlled oscillator is controlled by an input voltage input, and wherein the input voltage estimates an instantaneous oscillation frequency, and wherein the voltage controlled oscillator modulates signals applied to control the input thereby causing frequency modulation or phase modulation, and wherein the system produces continuous ultrasound waves at preset frequency with different power output levels.

2. The system according to claim 1, wherein the round bottomed flask has a volume of 100 ml and wherein the round bottomed flask is preferably made of glass.

3. The system according to claim 1, wherein the plurality of piezoelectric transducers is two, and wherein the piezoelectric transducers have a length of 12.1 mm, and wherein the piezoelectric transducers have a diameter of 20 mm, and wherein the two piezoelectric transducers are mounted 180° degrees apart near a central portion of the round bottomed flask, and wherein the piezoelectric transducers are mounted on the round bottomed flask using a dry epoxy material.

4. The system according to claim 1, wherein the system produces ultrasound waves of 29 kHz with different levels of power output, and wherein the spatial peak intensity (Isp) of the system is in a range of 4-455 mW/cm³.

5. A method of synthesizing taxol in a spherical bioreactor from hazel (Corylus avellana) cell culture, the method comprises the steps of:
obtaining the hazel (Corylus avellana) seeds tissue for cell line culture;
establishing rapid growing cell line culture or callus culture from hazel seed tissue in a modified Murashige and Skoog medium (MS medium);
establishing a suspension culture after/from cell line culture, and wherein the suspension culture from hazel seeds is established after growing the cell line culture or callus culture in modified the Murashige and Skoog medium (MS medium);
transferring the cells in a logarithmic growth phase along with culture media to a spherical ultrasonic bioreactor;
conducting preliminary studies for estimating an ultrasound dosage and time for treating suspension culture with ultrasound signals;
treating or irradiating the cell line culture in the spherical ultrasonic bioreactor with the ultrasound of preset power levels for preset time periods;
harvesting the cells after the ultrasound exposure;
washing and transferring the harvested cells to a fresh culture media for 1 week;
analysing the cells and culture media for molecular and biochemical parameters, wherein the cell suspension culture of the hazel (Corylus avellana) are analyzed for the molecular and biochemical parameters, and wherein the molecular and biochemical parameters are the factors influencing taxane production analysis;
drying the cells for extracting taxol; and
extracting intracellular and extracellular taxanes.

6. The method according to claim 5, wherein a modified Murashige and Skoog (MS) medium comprises an ammonium nitrate (NH₄NO₃) at a quantity of 1650 mgL⁻¹, monopotassium phosphate (KH₂PO₄) at a quantity of 170 mg L⁻¹, calcium chloride (CaCl₂) at a quantity of 332.02 mg L⁻¹, magnesium sulfate (MgSO₄) at a quantity of 180.54 mgL⁻¹, ferric-ethylenediamine-tetra-acetic acid (Fe-EDTA) at a quantity of 36.70 mgL⁻¹, boric acid (H₃BO₃) at a quantity of 6.20 mgL⁻¹, cupric sulfatepentahydrate (CuSO₄ 5H₂O) at a quantity of 0.025 mgL⁻¹, manganese(II) sulfate monohydrate (MnSO₄H₂O) at a quantity of 16.90 mg L⁻¹, Sodium molybdate dehydrate (Na₂MoO₄ 2H₂O) at a quantity of 0.25 mg L⁻¹, zinc sulfateheptahydrate (ZnSO₄7H₂O) at a quantity of 8.60 mg L⁻¹, potassium iodide (KI) at a quantity of 0.83, Cobalt(II) chloride hexahydrate (CoCl₂6H₂O) at a quantity of 0.025 mg L⁻¹, and supplemented with 3% sucrose, and a 4-dichlorophenoxyacetic acid at a quantity of 1 mg L⁻¹ and benzyladenine at a quantity of 0.5 mg L⁻¹, and wherein a pH of the modified Murashige and Skoog medium (MS medium) is 5.5.

7. The method according to claim 5, wherein the modified Murashige and Skoog medium (MS medium) for suspension culture of hazel seeds further comprises of a naphthalene acetic acid (NAA) at a quantity of 3 mg L⁻¹ and a indole-3-acetic acid (IAA) at a quantity of 3 mg L⁻¹.

8. The method according to claim 5, wherein 50 subculture of the cell line a suspension culture is established, and wherein the cells in suspension culture are incubated at 25° C. in dark with a shaking or agitation at 110 rpm on shaker incubator, and wherein the cells are sub-cultured every 7 days.

9. The method according to claim 5, wherein the cells are exposed to ultrasound of definite power intensities and exposure time periods, and wherein the preset power level of ultrasound for irradiating cell suspension culture is within a range of 4 to 455 mW/cm³, and wherein the preset time duration of ultrasound radiation for cell suspension culture is within a range of 2 to 60 min.

10. The method according to claim 5, wherein after ultrasound exposure the cells are harvested, cultured and transferred to a fresh culture media, and wherein the fresh culture media is a modified Murashige and Skoog medium (MS medium) for cell growth and taxol production, and wherein the cells are cultured for 1 week in the modified Murashige and Skoog medium (MS medium).

11. The method according to claim 5, wherein the taxane production is analyzed by high performance liquid chromatography-mass spectrometry (HPLC-MS).

12. The method according to claim 5, wherein the step of analysing the cells and culture media for molecular and biochemical parameters influencing taxane production comprises hydrogen peroxide (H₂O₂) production analysis, enzyme and enzyme activity analysis, an agarose gel electrophoresis analysis, a taxane production analysis of cell suspension culture with and without exposure to ultrasound, a viability and growth analysis of the hazel (Corylus avellana) cells after subjecting the cells to ultrasound irradiation, analysis of the relation between duration of ultrasound sonication and production of iodine, and analysis of the relation between the spherical Bessel function and luminescence.

13. The method according to claim 5, wherein the enzymes analysed are a 1-deoxy-D-xylulose-5-phosphate reductoisomerase (DXR) and a phenylalanine ammonia lyase (PAL).

14. The method according to claim 5, wherein the hydrogen peroxide (H₂O₂) is produced by the cell line of hazel under control conditions in spherical bioreactor, and wherein the hydrogen peroxide (H₂O₂) is a reactive oxygen species, and wherein the hydrogen peroxide (H₂O₂) promotes a production of a taxol or taxanes, and wherein the hydrogen peroxide (H₂O₂) is a signalling molecule stimulating a gene expression and promotestaxol production molecular pathway.

15. The method according to claim 5, wherein the hazel cells are exposed to ultrasound irradiation for a time period of 20 minutes to achieve a highest 1-deoxy-D-xylulose-5-phosphate reductoisomerase (DXR) activity in a harvesting time of 6 hours, and wherein the hazel cells are exposed to ultrasound irradiation for a time period of 20 minutes to achieve a highest phenylalanine ammonia lyase (PAL) activity in a harvesting time of 24 hours.

16. The method according to claim 5, wherein a highest taxol content is produced after exposing the hazel cell culture to ultrasound irradiation for 20 minute, and wherein a dry weight of the taxol content produced is 6 mg/Kg, when the hazel cell culture is exposed to ultrasound irradiation for 20 minute.

17. The method according to claim 5, wherein the exposure of hazel cells to ultrasound in the ultrasound bioreactor, has no adverse effects on a cell viability, a cell growth and a cellular membrane integrity.

18. The method according to claim 5, wherein the exposure of hazel cells to ultrasound has positive effects on biomass yields and increased taxanes biosynthesis.

19. The method according to claim 5, wherein the intracellular and extracellular taxanes are a taxol, a 10-deacetyl baccatin III and a baccatin III.

20. The method according to claim 5, wherein the extraction of cell-associated intracellular and extracellular taxol comprises the steps of:
taking the cells from the culture medium;
washing the cells;
drying the cells for a predetermined period at a room temperature;
powdering the dried cells for 5 min at room temperature;
dissolving the powdered cells in 10 mL methanol to obtain a solution;
ultrasonicating the solution of powdered cell in methanol for 40 min to obtain a homogenate;
filtering the homogenate;
airdrying and re-dissolving the filterate in a mixture of a methylene chloride and a water, and wherein the methylene chloride and the water are mixed in a ratio of 1:1;
centrifuging the dissolved filterate at 5000 rpm to obtain a supernatant and a pellet;
eluting the supernatant to collect the methylene chloride phase;
air-drying and re-dissolving the methylene chloride phase in a 250 mL methanol;
filtering the dissolved methylene chloride phase in a methanol with a 0.45-mm syringe filter, and wherein the filterate is subjected to a high-performance liquid chromatography (HPLC) analysis;
extracting extracellular taxol (in the medium) with methylene chloride (1:1) in a separating funnel;
collecting the methylene chloride phase;
air drying and re-dissolving the methylene chloride phase in a 250 mL methanol;
filtering the methyl chloride and methanol with a 0.45-mm syringe filter, and wherein the filtrate is subjected to a high-performance liquid chromatography (HPLC) analysis, and wherein the HPLC system is equipped with a C-18 column, and wherein the taxol is eluted at a flow rate of 1 mL min⁻¹ methanol and water (45:55, v/v), and wherein the taxol is detected at 227 nm using an ultraviolet detector, and wherein a quantity of taxol produced is estimated by comparison of a retention time and peak area with that of a genuine standard.
